(19) United States
(12) Patent Application Publication
(10) Pub. No.: US 2017/0258452 A1
(43) Pub. Date: Sep. 14, 2017

(54) ULTRASONIC IMAGE PROCESSING DEVICE, ULTRASONIC MEASUREMENT APPARATUS, AND ULTRASONIC IMAGE PROCESSING METHOD

(71) Applicant: SEIKO EPSON CORPORATION, Tokyo (JP)

(72) Inventor: Jiro TSURUNO, Okaya-shi (JP)

(73) Assignee: SEIKO EPSON CORPORATION, Tokyo (JP)

(21) Appl. No.: 15/428,456

(22) Filed: Feb. 9, 2017

(30) Foreign Application Priority Data
Mar. 10, 2016 (JP) 2016-046653

(51) Int. Cl. A61B 8/08; A61B 6/03; A61B 6/00; A61B 8/00

(52) U.S. Cl. CPC A61B 8/5246; A61B 8/4483; A61B 6/03; A61B 6/461; A61B 8/4444

(57) ABSTRACT

An ultrasonic image processing device includes an image acquisition unit that acquires a plurality of internal tomographic images of a target object in a plane including a first direction, along a second direction intersecting the first direction, an image dividing unit that divides each of the internal tomographic images into a plurality of separate images with a normal line to the first direction, and acquires the separate images, and an image combining unit that extracts separate images corresponding to coordinates on a continuous line which is continued in a plane including the first direction and the second direction, from the plurality of separate images, and arranges and combines the separate images in order of coordinates along the continuous line so as to generate a combined tomographic image.

### BACKGROUND

1. Technical Field

The present invention relates to an ultrasonic image processing device, an ultrasonic measurement apparatus, an ultrasonic image processing method, and the like.

2. Related Art

In the related art, there is an ultrasonic measurement apparatus used for puncture work for inserting a puncture needle into a living body (refer to JP-A-2012-139437), In the ultrasonic measurement apparatus (ultrasonic diagnosis apparatus) disclosed in JP-A-2012-139437, an ultrasonic probe is pressed against a living body so that ultrasonic measurement (ultrasonic wave transmission process and reception process) is performed, and an obtained internal tomographic image is displayed on a display section. The ultrasonic probe can obtain a two-dimensional internal tomographic image (B-mode image) by scanning a target object in a two-dimensional manner, and is formed of a mechanical scan probe which can perform three-dimensional scanning (two-dimensional internal tomographic image) by swinging a transmission angle of an ultrasonic wave, The ultrasonic measurement apparatus includes an image generation unit, extracts an image in which the puncture needle is reflected from each internal tomographic image which is obtained through three-dimensional scanning, and displays an overlap image in which the extracted internal tomographic images overlap each other.

Consequently, an operator can check to what extent the puncture needle is inserted with respect to a target position from the overlap image displayed on a monitor.

However, during the puncture work, it is necessary that a direction of a blood vessel into which the puncture needle is inserted is accurately checked, and the puncture needle is inserted along the direction of the blood vessel.

In the ultrasonic measurement apparatus disclosed in JP-A-2012-139437 a plurality internal tomographic images are acquired by swinging a transmission direction of an ultrasonic wave, and a position of the puncture needle can be checked by overlapping internal tomographic images in which the puncture needle is reflected with each other, but a direction of a blood vessel cannot be detected. Therefore, in order to determine from which direction the puncture needle is inserted into a blood vessel, for example, it is necessary to check a position or the blood vessel or a direction of the blood vessel, for example, by the operator adjusting an attitude such as a position or an angle of the ultrasonic probe, In other words, since the operator simultaneously has to perform adjustment work of the ultrasonic probe, insertion work of the puncture needle, and checking work of an internal tomographic image, time and effort for the puncture work cannot be sufficiently reduced.

### SUMMARY

An advantage of some aspects of the invention is to provide an ultrasonic image processing device, an ultrasonic measurement apparatus, and an ultrasonic image processing method, capable of reducing time and effort for puncture work. An ultrasonic image processing device according to this application example includes an image acquisition unit that acquires a plurality of internal tomographic images of a target object in a plane including a first direction, along a second direction intersecting the first direction; an image dividing unit that divides each of the internal tomographic images into a plurality of separate images with a normal line to the first direction, and acquires the separate images; and an image combining unit that extracts separate images corresponding to coordinates on a continuous line which is continued in a plane including the first direction and the second direction, from the plurality of separate images, and arranges and combines the separate images in order of coordinates along the continuous line so as to generate a combined tomographic image. In the application example, the image acquisition unit acquires a plurality of internal tomographic images along the second. direction, and the image dividing unit divides each of the internal tomographic images into a plurality of images with a normal line to the first direction. In other words, in a case where the internal tomographic image is, for example, an image in a plane including a first direction and a third direction orthogonal to the first direction, each separate image is a rectangular image along the third direction, and such a separate image is generated for each coordinate in the first direction. Since a plurality of internal tomographic images are acquired along the second direction, a single separate image is generated so as to correspond to a coordinate x in the first direction and a coordinate y in the second direction. The image combining unit extracts a separate image corresponding to each coordinate (x, y) on a continuous line which is continued in a plane including the first direction and the second direction, and arranges and combines extracted separate images in order of coordinates along the continuous line. Consequently, it is possible to generate a combined tomographic image along the continuous line.

In the application example, for example, it is possible to generate a combined tomographic image of a target object (for example, a living body) corresponding to a desired position of the continuous line without an operator changing a position or an angle of an ultrasonic probe performing ultrasonic measurement. Consequently, for example, when puncture work is performed, it is possible to easily detect a line direction of a blood vessel, and it is possible to efficiently perform the puncture work and to improve a puncturing success ratio by arranging an insertion direction in which a puncture needle is inserted with the line direction of the blood vessel. In the ultrasonic image processing device according to the application example, it is preferable that the continuous line is a straight line.

In a case where puncture work is performed, generally, a linear puncture needle is inserted into an organ such as a blood vessel. In this case, a blood vessel which is a destination of the puncture needle is also linear, and an insertion direction of the puncture needle preferably matches or substantially matches the linear direction. In the application example, the image combining unit generates a combined tomographic image corresponding to the linear continuous line, and thus it is possible to appropriately determine a position where a blood vessel is located in a linear direction.

It is preferable that the ultrasonic image processing device according to the application example further includes a display control unit that displays the combined tomographic image combined by the image combining unit on a display section, the image combining unit generates a plurality of the combined tomographic images obtained when the continuous line is moved in a plane including the first direction and the second direction, and the display control unit displays the plurality of combined tomographic images on the display section. In the application example with this configuration a plurality of combined tomographic images obtained when the continuous line is moved in a plane including the first direction and the second direction are generated, and the plurality of combined tomographic images are displayed on the display section. As a method of displaying each combined tomographic image on the display section, the combined tomographic image may be displayed in an animation manner (displayed in real time) in conjunction with movement of the continuous line, and plurality of combined tomographic images may be displayed to be arranged on a single screen. Consequently, an operator can easily understand an internal structure of a living body as an operation target on the basis of a combined tomographic image at each position obtained when the continuous line is moved. It is preferable that the ultrasonic image processing device according to the application example further includes an image selecting unit that selects a predetermined combined tomographic image from among the plurality of combined tomographic and the display control of unit displays the combined tomographic image selected by the image unit.

In the application example with this configuration, as described above, a plurality of combined tomographic images obtained when the continuous line is moved are displayed on the display section, and, for example, if the image selecting unit selects a combined tomographic image on the basis of an operation or the like z from an operator, the display control unit displays the selected combined tomographic image on the display section, Consequently, it is possible to display a combined tomographic image at a position desired to be checked by an operator on the display section, Particularly, in a case where combined tomographic images are sequentially displayed in an animation manner (displayed in real time) in a predetermined cycle through switching therebetween, the combined tomographic image can be displayed on the display section at any timing during animation display.

It is preferable that the ultrasonic image processing device according to the application example further includes a section position display unit that displays a position of the continuous line corresponding to the combined tomographic image selected by the image selecting unit.

In the application example with this configuration, in a case where a combined tomographic image is selected by the image selecting unit, the section position display unit displays a position of the continuous line corresponding thereto, The section position display unit may display a position of the continuous line in a display region of a combined tomographic image displayed on the display section in an overlapping manner, may display a position of the continuous line in other display regions, and may alternately display a combined tomographic image and a position of the continuous line in a switching manner. A position of the continuous line may be displayed on a display device which is different from the display section displaying a combined tomographic image. For example, the continuous line may be displayed on a liquid crystal display provided on an upper surface of an ultrasonic probe performing ultrasonic measurement.

In the application example with the configuration described above, a section of a combined tomographic image displayed on the display section is displayed to correspond to a certain position, and thus an operator can more efficiently perform puncture work on the basis of the combined, tomographic image and the position.

In the ultrasonic image processing device according to the application example, it is preferable that the image combining unit generates the plurality of combined tomographic images obtained when the continuous line passing through a first point in a plane including the first direction and the second direction is rotated centering on the first point.

In the application example with this configuration, each combined tomographic image, obtained when the continuous line is rotated centering on the first point through which the continuous line passes, is generated. For example, in a case where the continuous line is rotated centering on a point which is not present on the continuous line, there is a probability that a region which is not scanned may be present in a predetermined region in a plane including the first direction and the second direction, and thus the detection accuracy of a blood vessel is reduced. In contrast, in the application example, it is possible to cover the predetermined region. Among a plurality of combined tomographic images, in a combined tomographic image in which a dimension of blood vessel in a long axis direction is the maximum, the continuous line may be determined as being substantially along a line direction of the blood vessel. Therefore, an operator can easily specify a line direction of a blood vessel on the basis of a combined tomographic image in which a dimension of a blood vessel in a long axis direction is the maximum, and can thus easily judge an insertion direction of a puncture needle in puncture work. In the ultrasonic image processing device according to the application example, it is preferable that the first point is a vertex of a predetermined rectangular region in the plane including the first direction and the second direction.

In the application example with this configuration, the continuous line is rotated with a vertex of a predetermined rectangular region in the plane including the first direction and the second direction as the first point . In other words, one end (first point) on the continuous line is fixed, and the other end is moved along an outer peripheral edge of the rectangular region. In this case, combined tomographic images in various directions can be obtained by changing a vertex serving as the rotation center (first point) as appropriate, and thus an operator can more accurately determine a line direction of a blood vessel.

In the ultrasonic image processing device according to the application example, it is preferable that the image combining unit generates the plurality of combined tomographic images obtained when the continuous line is rotated with each vertex of the rectangular region as the first point.

In the application example with this configuration, a plurality of combined tomographic images obtained when the continuous line is rotated with each vertex of the rectangular region as the rotation center are generated. Consequently, since each combined tomographic image can be acquired when the continuous line is rotated centering on each vertex, even if a line direction of a blood vessel is any direction, the continuous line close to the line direction of the blood vessel can be detected with high accuracy.

In the ultrasonic image processing device according to the application example, it is preferable that the image combining unit generates the plurality of combined tomographic images obtained when the continuous line is rotated with, as the first therebetween among vertices of a predetermined rectangular region in the plane including the first direction and the second direction.

In the application example with this configuration, the continuous line is rotated with two vertices having no diagonal relationship therebetween as the rotation center. In a case where the continuous line is rotated about vertices having a diagonal relationship therebetween, detectable line directions of a blood vessel are substantially the same as each other, and a line direction of a blood vessel cannot be appropriately searched unless scanning in which the continuous line is rotated centering on other vertices is performed. In a case where scanning is performed with all of four vertices as the rotation center, directions in which detectable line directions of a blood vessel are substantially the same as each other are scanned twice, and thus a measurement time increases. In contrast, in the application example, since a combined tomographic image obtained when the continuous line is rotated about vertices having no diagonal relationship therebetween is generated, a line direction of a blood vessel can be detected with high accuracy, and a measurement time can be reduced. In the ultrasonic image processing device according to the application example, it is preferable that, in a case where intersections between the continuous line and an outer peripheral edge of the rectangular region are set as a first intersection and a second intersection, the image combining unit rotates the continuous line by alternately replacing the first point with the first intersection and the second intersection.

In the application example with this configuration since the continuous line is moved by alternately replacing the first point serving as the rotation center with one end and the other end of the continuous line, a combined tomographic image can be smoothly displayed when the combined tomographic image is displayed in an animation manner (displayed in real time). For example, vertices of the predetermined rectangular region in a plane including the first direction and the second direction are set to a first vertex, a second vertex, a third vertex, and a fourth vertex in a clockwise direction. A continuous line passing through the first vertex and the second vertex is rotated with the first vertex as the rotation center until the fourth vertex is located on the continuous line. Here, if a continuous line passing through the first vertex and the second vertex is set again, and the continuous line is rotated with the second vertex as the rotation center until the third vertex is located on the continuous line, animation display of a combined tomographic image which is previously displayed is not continued to animation display of a combined tomographic image which is previously displayed later. In contrast, in the application example, the first point is alternately replaced with one end and the other end of the continuous line, and thus the entire rectangular region is scanned. For example, the continuous line passing through the first vertex and the second vertex is rotated with the first vertex as the rotation center until the fourth vertex is located on the continuous line, and is then moved with the fourth vertex as the rotation center until the third vertex is located on the continuous line. In this case, such discontinuity in the animation display is removed, and thus an operator can easily understand a position of the continuous line and more efficiently perform puncture work on the basis of a combined tomographic image displayed on the display section.

In the ultrasonic image processing device according to the application example, it is preferable that the image combining unit rotates the continuous line and then inverts a rotation direction.

In the application example with this configuration, in a case where a combined tomographic image is displayed in an animation manner, a direction of the combined tomographic image viewed from an operator and an actual direction of the continuous line are not inverted to each other, and an internal structure can be easily understood.

For example, in a rectangular region having a first vertex and a fourth vertex located on the left, a second vertex and a third vertex located on the right, when viewed from an operator, a continuous line passing through the first vertex and the second vertex is rotated with the first vertex as the rotation center until the fourth vertex is located on the continuous line. In this case, when viewed from the operator, a position of the left first vertex is also displayed to be located on the left in a combined tomographic image on the display section, and thus there is no feeling of incompatibility. On the other hand, if the continuous line is further rotated with the fourth vertex as the rotation center, a position corresponding to the fourth vertex is located on the right in the combined tomographic image displayed on the display section regardless of the fourth vertex being located on the left when viewed from the operator. Therefore, the left and right sides are inverted to actual ones, and thus it is hard to understand an internal structure.

In contrast, in the application example with the configuration described above, for example, the continuous line passing through the first vertex and the second vertex is rotated with the first vertex as the rotation center until the fourth vertex is located on the continuous line, then the rotation direction is inverted, and the continuous line is rotated until the second vertex is located on the continuous line. Next, the continuous line is rotated with the second vertex as the rotation center until the third vertex is located on the continuous line, then the rotation direction is inverted, and the continuous line is rotated until the first vertex is located on the continuous line. In this case, a position of the combined tomographic image displayed on the display section and an actual position of the continuous line are not horizontally inverted, and thus it is possible for an operator to easily understand a position of the continuous line with respect to the combined tomographic image and to more efficiently perform puncture work.

It is preferable that the ultrasonic image processing device according to the application example further includes a first point selecting unit that selects the first point on the continuous line, and, in a case where the first point is not an end of the continuous line, the image combining unit generates the plurality of combined tomographic images obtained when one of separate lines obtained by dividing the continuous line with respect to the first point is rotated centering on the first point.

In the application example with this configuration, the image combining unit divides the continuous line into separate lines centering on the first point selected by the first point selecting unit, and one of the separate lines is rotated centering on the first point. In this case, for example, if a blood vessel branches or bends in the middle, the first point is selected at a position corresponding to a branching point or a bending point, and thus it is possible to acquire a combined tomographic image along a line direction of a blood vessel. An ultrasonic measurement apparatus according to this application example includes an ultrasonic probe that acquires a plurality of internal tomographic images of a target object in a plane including a first direction, along a second direction intersecting the first direction through transmission and reception of an ultrasonic wave; an image acquisition unit that acquires the internal tomographic images from the ultrasonic probe; an image dividing unit that divides each of the internal tomographic images into a plurality or separate images with a normal line to the first direction, and acquires the separate images; and an image combining unit that extracts separate images corresponding to coordinates on a continuous line which is continued in a plane including the first direction and the second direction, from the plurality of separate images, and arranges and combines the separate images in order of coordinates along the continuous line so as to generate a combined tomographic image.

In the application example, in the same manner as in the above-described ultrasonic image processing device, it is possible to generate an internal tomographic image (combined tomographic image) of a target object (for example, a living body) corresponding to a desired position of the continuous line without an operator changing a position or an angle of an ultrasonic probe. Consequently, for example, when puncture work is performed, it is possible to easily arrange an insertion direction in which a puncture needle is inserted with the line direction of the blood vessel, and thus it is possible to efficiently perform the puncture work and to improve a puncturing success ratio.

In the ultrasonic measurement apparatus according to the application example, it is preferable that the ultrasonic probe includes a plurality of ultrasonic transducers that are disposed in an array form along the first direction and the second direction; a common electrode wiring that connects the ultrasonic transducers along the first direction to each other; a driving electrode wiring that connects the ultrasonic transducers along the second direction to each other; and a bias voltage output portion that outputs a bias voltage to the common electrode wiring, and the bias voltage output portion includes a voltage switching unit that switches between a first bias voltage causing reception of the ultrasonic wave to be valid and a second bias voltage causing reception of the ultrasonic wave to be invalid.

In the application example with this configuration, among a plurality of ultrasonic transducers disposed in an array form along the first direction and the second direction, the ultrasonic transducers along the first direction are connected to each other via the common electrode wiring, and the ultrasonic transducers along the second direction are connected to each other via the driving electrode wiring. In the ultrasonic probe, during an ultrasonic wave reception process of receiving an ultrasonic wave, the bias voltage output portion outputs the first bias voltage causing reception of the ultrasonic wave to be valid, to an ultrasonic transducer performing reception (from which a received signal is desired to be extracted), and outputs the second bias voltage causing reception of the ultrasonic wave to be invalid, to an ultrasonic transducer not performing reception (from which a received signal is not acquired).

In other words, in the ultrasonic wave reception process, the first bias voltage is output to ultrasonic transducers corresponding to a position (measurement region) where an internal tomographic structure of a living body is desired to be measured, and the second bias voltage is output to the other ultrasonic transducers. Consequently, in the ultrasonic transducers corresponding to regions other than the measurement region, the reception sensitivity is low and thus an output received signal is also reduced. On the other hand, in the ultrasonic transducers corresponding to the measurement region, the reception sensitivity is high, and a received signal corresponding to an internal tomographic structure can be appropriately obtained.

In the ultrasonic probe, by switching between the common electrode wirings to which the first bias voltage and the second bias voltage are output, it is possible to perform ultrasonic measurement (an ultrasonic wave transmission process and an ultrasonic wave reception process) by using ultrasonic transducers corresponding to the measurement region among the ultrasonic transducers disposed in a two-dimensional array structure. For example, if the first bias voltage is output to common electrode wirings corresponding to a first measurement region, and the second bias voltage is output to of r common electrode wirings, an internal tomographic structure corresponding to the first measurement region can be measured. By switching between output destinations of the bias voltages so that the first bias voltage is output to common electrode wirings corresponding to a second measurement region which is different from the first measurement region, and the second bias voltage is output to other common electrode wirings, an internal tomographic structure corresponding to the second measurement region can be measured. Consequently, the image acquisition unit can measure internal tomographic structures at a plurality of positions.

An ultrasonic image processing method according to this application example includes acquiring a plurality of internal tomographic images of a target object in a plane including a first direction, along a second direction intersecting the first direction; dividing each of the internal tomographic images into a plurality of separate images with a normal line to the first direction, and acquiring the separate images; and extracting separate images corresponding to coordinates on a continuous line which is continued in a plane including the first direction and the second direction, from the plurality of separate images, and arranging and combining the separate images in order of coordinates along the continuous line so as to generate a combined tomographic image.

In the application example, in the same manner as in the above-described ultrasonic image processing device, it is possible to generate an internal tomographic image (combined tomographic image) of a target object (for example, a living body) corresponding to a desired position of the continuous line without an operator changing a position or an angle of an ultrasonic probe . Consequently, for example, when puncture work is performed, it is possible to easily arrange an insertion direction in which a puncture needle is inserted with the line direction of the blood vessel, and thus it is possible to efficiently perform the puncture work and to improve a puncturing success ratio.

### BRIEF DESCRIPTION OF THE DRAWINGS

The invention will be described with reference to the accompanying drawings, wherein like numbers reference like elements.

FIG. 1 is a block diagram illustrating a schematic configuration of an ultrasonic measurement apparatus according to the first embodiment.

FIG. 2 is a perspective view illustrating a schematic configuration of an ultrasonic probe of the first embodiment.

FIG. 3 is a plan view illustrating a schematic configuration of an ultrasonic sensor of the first embodiment.

FIG. 4 is an enlarged plan view of the ultrasonic sensor obtained by partially enlarging the ultrasonic sensor in FIG. 3.

FIG. 5 is a schematic sectional view of the ultrasonic sensor taken along a line A-A in FIG. 4.

FIG. 6 is a block diagram schematically illustrating a circuit configuration of the ultrasonic probe of the first embodiment.

FIG. 7 is a diagram illustrating a relationship between a first bias voltage and a second bias voltage.

FIG. is an image diagram illustrating a case where an ultrasonic measurement process is performed on a living body by using the ultrasonic probe of the first embodiment.

FIG. 9 is a flowchart illustrating an ultrasonic measurement method in the first embodiment.

FIG. 10 is a flowchart illustrating an ultrasonic image acquisition process in FIG. 9.

FIG. 11 a timing chart illustrating the ultrasonic measurement process of the first embodiment.

FIG. 12 is a diagram for explaining a driving order of element portions in an ultrasonic measurement method of the first embodiment.

FIG. 13 is a diagram illustrating examples of separate images generated from an internal tomographic image in the first embodiment.

FIG. 14 is a flowchart illustrating a combined image display process in FIG. 9.

FIG. 15 is a diagram illustrating an example of a combined tomographic image generated from separate images in the first embodiment.

FIG. 16 is a diagram illustrating an example of a combined tomographic image displayed in a display region of a display section in the first embodiment.

FIG. 17 is a diagram for explaining movement procedures of a continuous line in the first embodiment.

FIG. 18 is a diagram illustrating an example of transition of a combined tomographic image displayed on the display section in the first embodiment.

FIG. 19 is a flowchart illustrating a second ultrasonic measurement process in the first embodiment.

FIG. 20 is a diagram for explaining movement procedures of rotating the entire continuous line of the first embodiment centering on a first point.

FIG. 21 is a diagram for explaining movement procedures of the continuous line in a case where the continuous line of the first embodiment is divided with the first point as a boundary.

FIG. 22 is a diagram for explaining movement procedures of a continuous line in a second embodiment.

FIG. 23 is a diagram for explaining movement procedures of a continuous line in a third embodiment.

FIG. 24 is a diagram illustrating other examples of movement procedures of a continuous line.

FIG. 25 is a diagram illustrating still other examples of movement procedures of a continuous line.

### DESCRIPTION OF EXEMPLARY EMBODIMENTS

### First Embodiment

Hereinafter, a description will be made of an ultrasonic measurement apparatus according to a first embodiment.

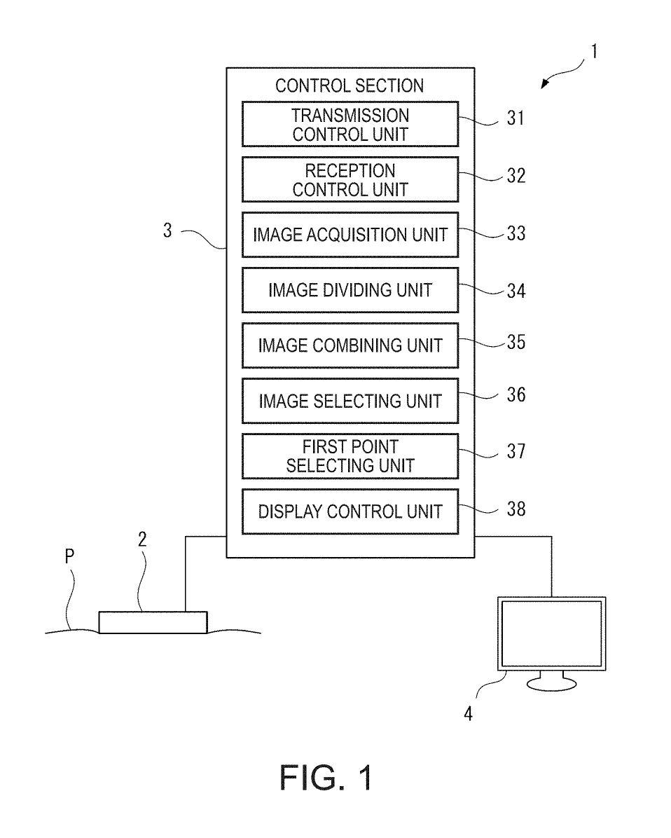

FIG. 1 is a block diagram illustrating a schematic configuration of an ultrasonic measurement apparatus 1 according to the first embodiment.

As illustrated in FIG. 1, the ultrasonic measurement apparatus 1 of the present embodiment includes an ultrasonic probe 2 fixed to a target object (a living body P in the present embodiment), a control section 3 which controls the ultrasonic probe 2 to obtain an internal tomographic image of the living body P, and a display section 4 which displays the obtained internal tomographic image.

The ultrasonic measurement apparatus 1 of the present embodiment can be appropriately used to perform, for example, puncture work for inserting a puncture needle 11 (refer to FIG. 8) into a predetermined organ (for example, a blood vessel) of the living body P. In the subsequent description, a description will be made of a case where the ultrasonic measurement apparatus 1 is used for the puncture work as an example, but the ultrasonic measurement apparatus 1 is not limited to the puncture work, and may be used to perform ultrasonic diagnosis on an affected part position of the living body P.

In puncture work, the ultrasonic measurement apparatus 1 performs an ultrasonic wave transmission process in which the ultrasonic probe 2 is fixed to an affected part position of the living body P on which puncture is desired to be performed, and an ultrasonic wave is transmitted into the living body P from the ultrasonic probe 2, and an ultrasonic wave reception process in which a reflected ultrasonic wave reflected inside the living body P is received. The ultrasonic probe 2 outputs a received signal obtained through the ultrasonic wave reception process, to the control section 3 The control section 3 forms an internal tomographic image of the living body P on the basis of the received signal, and displays the internal tomographic image on the display section 4.

By using the ultrasonic measurement apparatus 1, an operator can efficiently perform puncture work while checking (observing) the internal tomographic image displayed on the display section 4.

Hereinafter, each configuration of the ultrasonic measurement apparatus 1 of the present embodiment will be described in detail.

Ultrasonic Probe

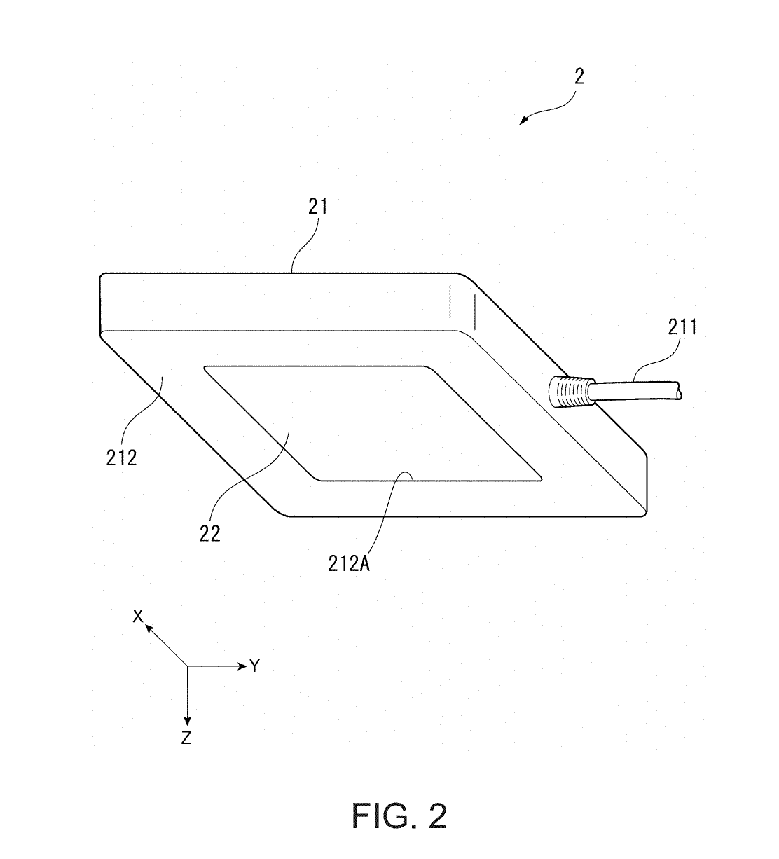

FIG. 2 is a perspective view illustrating a schematic configuration of the ultrasonic probe 2 of the first embodiment.

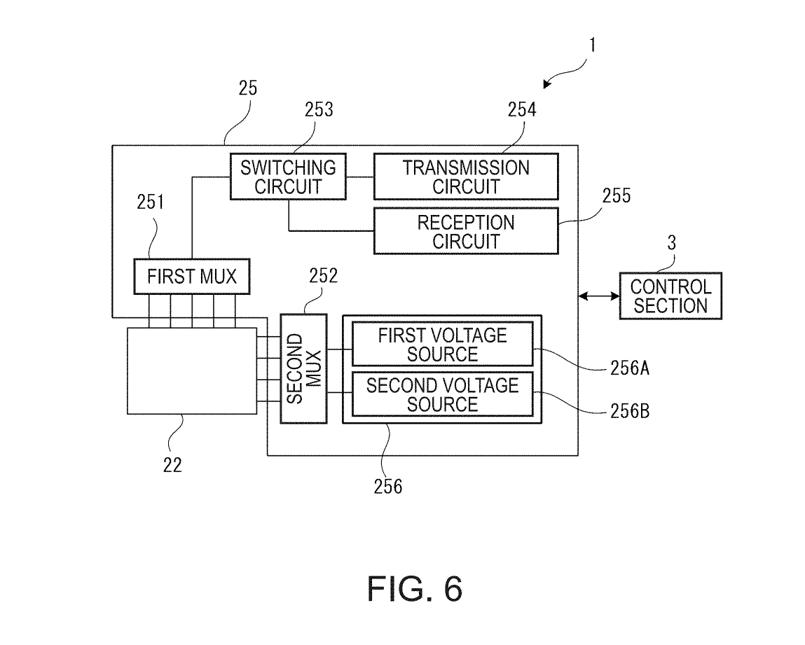

The ultrasonic probe 2 of the present embodiment is configured to include, as illustrated in FIG. 2, a casing 21, an ultrasonic sensor 22 stored in the casing 21, and a circuit board 25 (refer to FIG. 6). The ultrasonic probe 2 is connected to the control section 3 via, for example, a signal cable 211, and thus the ultrasonic probe 2 and the control section 3 are communicably connected to each other.

The casing 21 is, for example, a rectangular box-shaped member in a plan view, and stores the ultrasonic sensor 22 or the circuit board 25 therein. The casing 21 is provided with a sensor window 212A on one surface (sensor surface 212) facing the living body P, and the ultrasonic sensor 22 is provided in the sensor window 212A so as to face the outside (the living body P side).

When puncture work is performed, the ultrasonic probe 2 is fixed to the living body P via an adhesion layer (not illustrated) At this time, an acoustic matching agent such as a gel is filled between the ultrasonic sensor 22 exposed from the sensor window 212A and the living body P, and an ultrasonic wave propagates between the ultrasonic sensor 22 and the living body P with high efficiency.

Ultrasonic Sensor

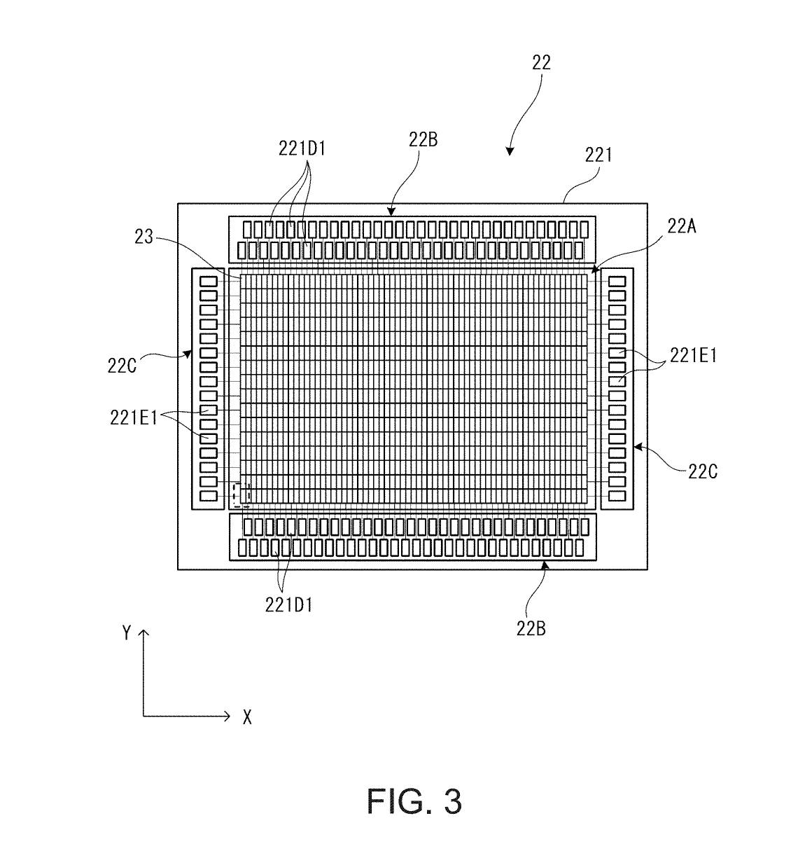

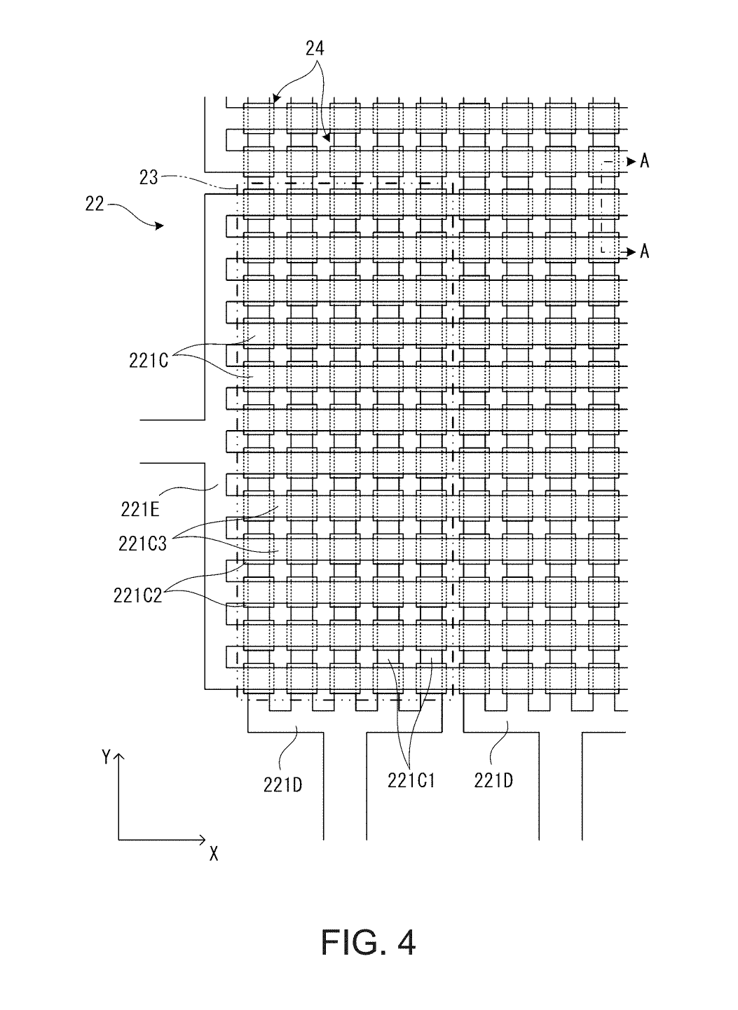

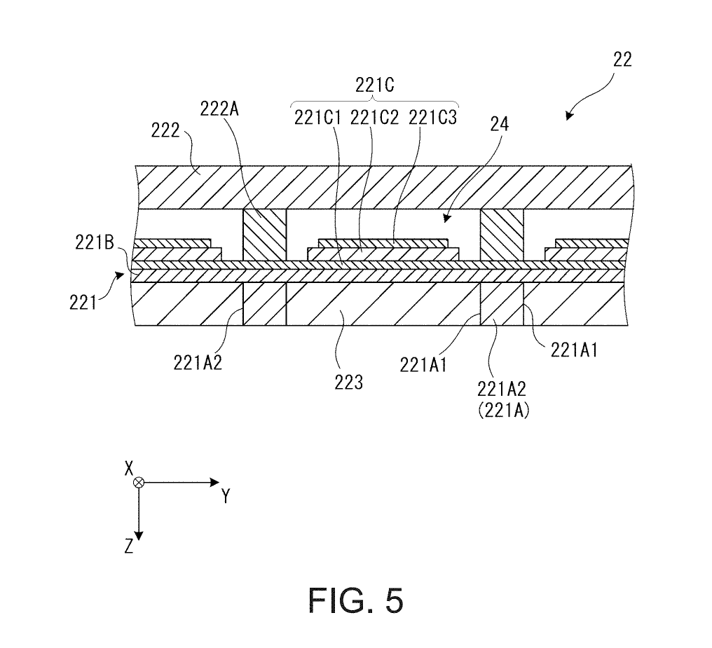

Next, the ultrasonic sensor 22 will be described. FIG. 3 is a plan view illustrating a schematic configuration of the ultrasonic sensor 22 of the present embodiment. FIG. 4 is an enlarged plan view obtained by enlarging the ultrasonic sensor 22 illustrated in FIG. 3. FIG. 5 is a schematic sectional view of the ultrasonic sensor 22 taken along the line A-A in FIG. 4. In FIGS. 3 and 4, a sealing plate 222 is not illustrated.

As illustrated in FIG. 3, the ultrasonic sensor 22 includes an array region 22A, a driving terminal region 22B, and a common terminal region 22C.

A plurality of element portions 23 which are disposed in a two-dimensional array form in an X direction (first direction) and a Y direction (second direction) intersecting (in the present embodiment, for example, orthogonal to) each other are provided in the array region 22A. Each of the element portions is configured to include a predetermined number of ultrasonic transducers 24 which are disposed in an array form along the X direction and the Y direction as illustrated in FIG. 4.

In other words, the element portion 23 is configured to include m×n ultrasonic transducers 24 of m (m=5 in the example illustrated in FIG. 4) in the X direction and n (n−12 in the example illustrated in FIG. 4) in the Y direction, and the ultrasonic sensor 22 is formed of M×N element portions 23 of M (in the present embodiment, M=64) in the X direction and N (in the present embodiment, N−16) in the direction. The ultrasonic sensor 22 as described above is configured to include, for example, as illustrated in FIG. 5, an element board 221, the sealing plate 222, an acoustic matching layer 223, and the like.

As illustrated in FIG. 5, the element board 221 includes a base 221A, a vibration film 221B, and a piezoelectric element 221C. The base 221A is formed of, for example, a semiconductor substrate such as Si. The base 221A is provided with an opening 221A1 corresponding to each ultrasonic transducer 24. In the present embodiment, each opening 221A1 is a through hole which penetrates through the base 221A in a substrate thickness direction, and the vibration film 221B is provided on one end side (sealing plate 222 side) of the through hole.

The vibration film 221B is made of, for example, SiO₂ or a laminate of SiO₂ and ZrO₂, and is provided to entirely cover the base 221A on the sealing plate 222 side. In other words, the vibration film 221E is supported by a partition wall 221A2 forming the opening 221A1, and closes the opening 221A1 on the sealing plate 222 side. A thickness dimension of the vibration film 221B is sufficiently smaller than a thickness dimension of the base 221A.

The piezoelectric element 221C is provided on the vibration film 221B closing each opening 221A1 as illustrated in FIGS. 4 and 5. The piezoelectric element 221C is formed of a lower electrode 221C1, a piezoelectric film 221C2, and an upper electrode 221C3. Here, a single ultrasonic transducer 24 is formed of a region of the vibration film 221B closing the opening 221A1 and the piezoelectric element 221C.

In the ultrasonic transducer 24, a rectangular wave voltage having a predetermined frequency is output between the lower electrode 221C1 and the upper electrode 221C3, so that the piezoelectric film 221C2 is deformed, and thus the vibration film 221B closing the opening 221A1 vibrates. Therefore, an ultrasonic wave is transmitted (ultrasonic wave transmission process). If an ultrasonic wave is input to the vibration film 221B, and thus the vibration film 221B vibrates, a potential difference is generated between the lower electrode 221C1 side and the upper electrode 221C3 side of the piezoelectric film 221C2. Consequently, a potential difference between the lower electrode 221C1 and the upper electrode 221C3 is detected, and thus it is possible to detect that the ultrasonic wave is received (ultrasonic wave reception process).

In the present embodiment, as described above, the ultrasonic transducers 24 are disposed along an array form in the X direction and the Y direction.

Here, the lower electrode 221C1 is a driving electrode wiring, and is linearly formed along the Y direction, A plurality of lower electrodes 221C1 are arranged in parallel along the X direction. In other words, the lower electrodes 221C1 are provided to cross the plurality of ultrasonic transducers 24 arranged in the Y direction, and thus connect the ultrasonic transducers 24 to each other. In the present embodiment, a single element portion 23 is formed of m ultrasonic transducers 24 in the X direction and n ultrasonic transducers 24 in the Y direction, and the lower electrodes 221C1 connect the ultrasonic transducers 24 forming the element portion 23 to each other. The lower electrode 221C1 has a linear shape along the Y direction as described above, and crosses the M element portions 23 arranged in the Y direction. That is, the element portions 23 arranged in the Y direction are connected to each other via the lower electrode 221C1.

Specifically, the m lower electrodes 221C1 arranged along the X direction are connected to each other via driving connection lines 221D at both ends thereof in the Y direction, A part of each of the driving connection line 221D extends to the driving terminal region 22B along the Y direction, and is provided with a driving terminal 221D1 (SIG terminal) connected to the circuit board 25 at a front end thereof as illustrated in FIG. 3.

On the other hand, the upper electrode 221C3 is a common electrode wiring, and is linearly formed along the X direction. A plurality of upper electrodes 221C3 are arranged in parallel along the Y direction. In other words, the upper electrodes 221C3 are provided to cross the plurality of ultrasonic transducers 24 arranged in the X direction, and thus connect the ultrasonic transducers 24 to each other.

The upper electrodes 221C3 connect the ultrasonic transducers 24 forming a single element portion 23 to each other. The upper electrodes 221C3 has a linear shape along the X direction as described above, and crosses the N element portions 23 arranged in the X direction. That is, the element portions 23 arranged in the X direction are connected to each other via the upper electrodes 221C3.

Specifically, the n upper electrodes 221C3 arranged along the Y direction are connected to each other via common connection lines 221E at both ends thereof in the X direction. A part of each of the common connection line 221E extends to the common terminal region 220 along the X direction, and is provided with a common terminal 221E1 (COM terminal) connected to the circuit board 25 at a front end thereof.

Next, the sealing plate 222 forming the ultrasonic sensor 22 will be described. The sealing plate 222 is bonded to the element board 221 so as to reinforce the element board 221. The sealing plate 222 is formed to cover the region of the element board 221 in which the ultrasonic transducers 24 are disposed in a plan view viewed from the Z direction, and is formed of a semiconductor substrate such as Si or an insulator substrate. A material or a thickness of the sealing plate 222 influences frequency characteristics of the ultrasonic transducer 24, and is thus preferably set on the basis of a center frequency of an ultrasonic wave which is transmitted and received in the ultrasonic transducer 24.

The sealing plate 222 is bonded to the element board 221 via, for example, a bonding film 222A which is formed on the vibration film 221E of the element board 221. The bonding film 222A is provided to correspond to a region (the partition wall 221A2 between the openings 221A1) other than the opening 221A1 of the base 221A. Therefore, vibration of the vibration film 221B is not hindered by the bonding film 222A, and crosstalk between the respective ultrasonic transducers 24 can be reduced.

Although not illustrated, the sealing plate 222 is provided with a through hole so as to oppose a terminal of the lower electrode 221C1 or the upper electrode 221C3, and an electrode connecting the lower electrode 221C1 or the upper electrode 221C3 to the circuit board 25 is provided in the through hole. As the electrode, for example, a through electrode may be used, and a lead wire or an FPC may be used.

The acoustic matching layer 223 is provided on an ultrasonic wave transmission/reception side of the element board 221 so as to be embedded in the opening 221AI of the base 221A as illustrated in FIG. 5.

The acoustic matching layer 223 causes an ultrasonic wave transmitted from the ultrasonic transducer 2 to propagate through the living body P, and causes an ultrasonic wave reflected inside the living body P to efficiently propagate toward the ultrasonic transducer 24. Thus, the acoustic matching layer 223 is required to be set to have an intermediate acoustic impedance between the acoustic impedance of the ultrasonic transducer 24 and the acoustic impedance of the living body P. A material having such an acoustic impedance may be, for example, silicon.

Circuit Board

Next, the circuit board 25 will be described.

FIG. 6 is a block diagram illustrating a schematic circuit configuration of the ultrasonic probe 2 of the present embodiment.

The circuit board 25 is configured to include a first multiplexer (first MUX 251), a second multiplexer (second MUX 252), a switching circuit 253, a transmission circuit 254, a reception circuit 255, and a voltage source 256.

The first MUX 251 is connected to the respective driving terminals 221D1 of the, driving terminal region 221B and the switching circuit 253, The first MUX 251 switches between the driving terminals 221D1 which output a drive voltage (drive signal) or incorporate a received signal on the basis of the control of the control section 3.

The second MUX 252 is connected to the respective common terminals 221E1 of the common terminal region 22C and the voltage source 256. The second MUX 252 is a voltage switching portion, and forms a bias voltage output portion along with the voltage source 256 which will be described later. In other words, the second MUX 252 switches between the common terminals 221E1 which output a voltage which is output from the voltage source 256, on the basis of the control of the control section 3. Specifically, a first bias voltage V1 and a second bias voltage V2 are input to the second MUM 252 from the voltage source 256. The second MUM 252 outputs the first bias voltage V1 to the common terminal 221E1 connected to the element portion 23 which is a received signal acquisition target, and outputs the second bias voltage V2 to the other common terminals 221E1, on the basis of the control of the control section 3.

The switching circuit 253 switches between transmission connection for connecting the driving terminal 221D1 to the transmission circuit 254, and reception connection for connecting the driving terminal 221D1 to the reception circuit 255 under the control of the control section 3.

The transmission circuit 254 has pulsation for outputting a pulsed drive signal. In an ultrasonic wave transmission process, the transmission circuit 254 outputs a drive signal to the driving terminal 221D1 via the switching circuit 253 and the first MUX 251 when the switching circuit 253 performs switching to transmission connection. In the present embodiment, a predetermined voltage (for example, 15 V) is normally applied to the driving terminal 221D1, and thus the drive signal superimposed on the voltage is output.

In the ultrasonic wave reception process, when the switching circuit 253 performs switching to reception connection, a received signal from the driving terminal 221D1 is input to the reception circuit 255, The reception circuit 255 is configured to include, for example, a linear noise amplifier, an A/D converter, and the like, and performs various signal processes such as conversion of the input received signal into a digital signal, removal of a noise component, amplification to a desired signal level, and a phasing addition process, and outputs the processed received signal to the control section 3.

The voltage source 256 includes a first voltage source 256A and a second voltage source 256B.

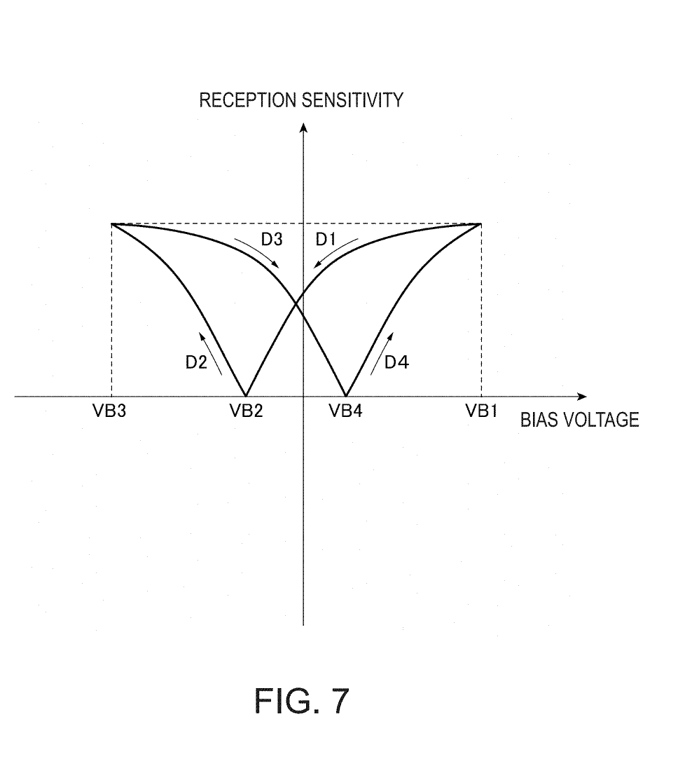

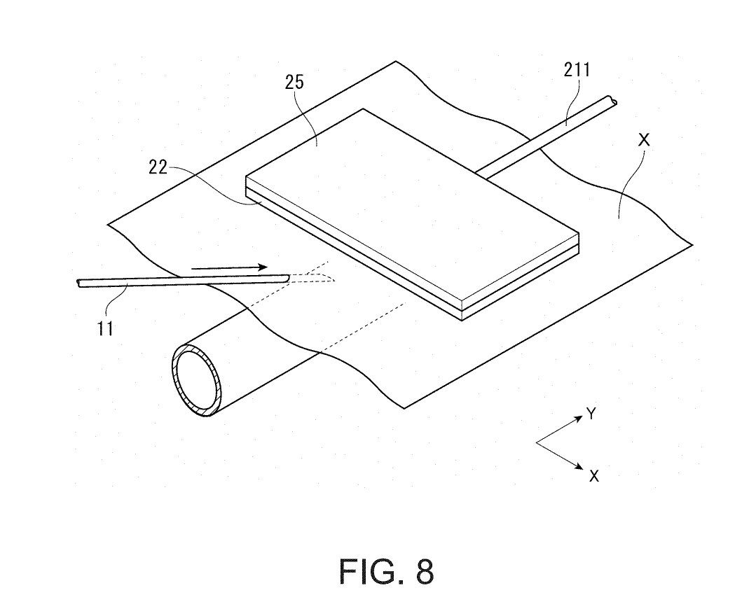

The first voltage source 256A generates the first bias voltage V1 which will be output to the common terminal 221E1, and outputs the, first bias voltage V1 to the second MUX 252. The second voltage source 256B generates the second bias, voltage V2 which will be output to the common terminal 221E1, and outputs the second bias voltage V2 to the second MUX 252. FIG. 7 is a diagram illustrating a relationship between the first bias voltage V1 and the second bias voltage V2.

In the present embodiment, in a case where an ultrasonic wave is received, a bias voltage is output to the upper electrode 221C3 (common terminal 221E1), and a potential difference generated in the piezoelectric film 22102 when the vibration film 221B vibrates is extracted from the driving terminal 221D1 connected to the lower electrode 221C1. The bias voltage is a difference between a voltage output to the driving terminal 221D1 and a voltage output to the common terminal 221E1. For example, in a case where a voltage of +15 V is output to the driving terminal 221D1, and a voltage of 18 V is output to the common terminal 221E1, the bias voltage is −3 V.

Here, as illustrated in FIG. 7, the reception sensitivity of an ultrasonic wave in the ultrasonic transducer 24 changes depending on a bias voltage.

If a bias voltage is reduced from VB1 at which the reception sensitivity is the maximum, the reception sensitivity gradually decreases as in an arrow D1 in FIG. 7, and the reception sensitivity becomes nearly 0 when the bias voltage is DB2. If the bias voltage is further reduced, a phase of a received signal is inverted, the reception sensitivity increases again as indicated by an arrow D2, and the reception sensitivity becomes the maximum when the bias voltage is VB3. Absolute values of the reception sensitivity at VB1 and VB3 are substantially the same as each other, but a phase of a received signal is inverted.

If the bias voltage is gradually increased from VB3, as indicated by an arrow D3 in FIG. 7, the reception sensitivity gradually decreases, and the reception sensitivity becomes nearly 0 when the bias voltage is VB4. If the bias voltage is further increased, a phase of a received signal is inverted, the reception sensitivity increases again as indicated by an arrow D4, and the reception sensitivity becomes the maximum when the bias voltage is VB1.

Here, in the present embodiment, the first bias voltage V1 is a voltage output to the common terminal 221E1 so that the maximum reception sensitivity is obtained, and a difference between a voltage output to the driving terminal 221D1 and the first bias voltage V1 is VB1. The first bias voltage V1 is output to the common terminal 221E1 even when an ultrasonic wave is transmitted.

The second bias voltage V2 is a voltage output to the common. terminal 221E1 when the reception sensitivity initially becomes 0 (or the reception sensitivity is in a predetermined range centering on 0) as a result of reducing a voltage from the first bias voltage V1. A difference between a voltage output to the driving terminal 221D1 and the first bias voltage V1 is VB2.

Control Section

Next, a description will be made of the control section the ultrasonic measurement apparatus 1.

The control section 3 is an ultrasonic image processing device, and is configured to include a calculation unit formed of a central processing unit (CPU) and the like, and a storage unit formed of a memory and the like.

The storage unit stores various programs or various data for performing ultrasonic measurement using the ultrasonic probe 2, or generation and display of an internal tomographic image of the living body P based on an ultrasonic measurement result. By reading and executing the various programs stored in the storage unit, the calculation unit functions as a transmission control unit 31, a reception control unit 32, an image acquisition unit 33, an image dividing unit 34, an image combining unit 35, an image selecting unit 36, a first point selecting unit 37, a display control unit 38, and the like, as illustrated in FIG. 1. The control section 3 may be provided with an operation input unit formed of a keyboard and the like. The transmission control unit 31 controls the ultrasonic probe 2, so as to transmit ultrasonic waves from the ultrasonic transducers 24 included in a predetermined element portion 23 of the ultrasonic sensor 22. Specifically, the transmission control unit 31 causes the switching circuit 253 to perform switching to transmission connection, so that the first bias voltage V1 is output to each common terminal 221E1 from the voltage source 256, and a drive voltage (drive signal) based on a pulse signal from the transmission circuit 254 is output to a predetermined driving terminal 221D1.

The reception control unit 32 controls the ultrasonic probe 2 to receive a received signal from a predetermined element portion 23 of the ultrasonic sensor 22. Specifically, the reception control unit 32 causes the switching circuit 253 to perform switching to reception connection, so that the first bias voltage V1 is output from the voltage source 256 to the common terminal 221E1 corresponding to the element portion 23 which is a received signal acquisition target, and the second bias voltage V2 is output to the other common terminals 221E1. The received signal output from the driving terminal 221D1 corresponding to the element portion 23 which is a received signal acquisition target is acquired via the reception circuit 255.

The image acquisition unit 33 acquires the received signal (image signal) transmitted from the ultrasonic probe 2, and generates (acquires) an internal tomographic image at each position of the living body P.

The image dividing unit 34 divides each acquired internal tomographic image into a plurality of images with a normal line (a straight line along the Z direction) orthogonal to the X direction, and sets the plurality of images as a plurality of separate images each of which is rectangular in the Z direction. The number of separate images obtained through division in the image dividing unit 34 is not particularly limited, but a separate image is preferably generated for each X coordinate. For example, in a case where an image size of an internal tomographic image is X_{M}×Z_{M}, the image dividing unit 34 generates X_{M} separate images each having an image size of 1×Z_{M} according to respective pixels (1≦x≦X_{M}) in the X direction. A plurality of (for example, Y_{M}) internal tomographic images along the Y direction are acquired. In the present embodiment, coordinates y=1 to Y_{M} indicating Y positions are added to the internal tomographic images in order from a −Y side. Consequently, each separate image obtained by dividing each internal tomographic image can be expressed by coordinates in an XY plane. In the following description, a separate image (s, t) indicates a separate image at a position of x=s in an internal tomographic image of y=t.

The image combining unit 35 combines a plurality of separate images so as to generate a combined tomographic image. Specifically, the image combining unit 35 extracts separate images (s, t) corresponding to coordinates on a predetermined continuous line in the XY plane, and joins the separate images in order of the coordinates of the continuous line so as to generate a combined tomographic image The image combining unit 35 generates each combined tomographic image obtained when the continuous line is moved in the XY plane. The continuous line is a straight line in the present embodiment, and indicates a section position corresponding to a combined tomographic image.

In a case where an operation signal indicating that an operator of the ultrasonic measurement apparatus I operates an operation input unit so as to select an image is input, the image selecting unit 36 selects a combined tomographic image.

In other words, in the present embodiment, the image combining unit 35 generates a combined tomographic image obtained when the continuous line is moved, in a predetermined cycle, and displays the generated combined tomographic image on the display section 4 in real time. In other words, combined tomographic images obtained when the continuous line is moved in the XY plane are displayed in an animation manner. When an input operation for selecting an image is input at a predetermined timing during animation display, the image selecting unit 36 selects a combined tomographic image which is being displayed on the display section 4 at the timing. The selected combined tomographic image may be displayed on the display section 4 immediately after being selected, and a predetermined number of images may be selected, and then the selected images may be collectively displayed on the display section 4.

In a case where the operator operates the operation input unit so as to set a position with respect to an internal tomographic image or a combined tomographic image displayed on the display section 4, the first point selecting unit 37 selects (x,y) coordinate position of a separate image corresponding to the position.

The display control unit 38 displays an internal tomographic image or a combined tomographic image on the display section 4. The display control unit 33 displays a position (a position of a continuous line) of the internal tomographic image or the combined tomographic image in the XY plane on the display section 4.

A specific process of the control section 3 will be described later.

Ultrasonic Measurement Method

Next, a description will be made of an ultrasonic measurement method (ultrasonic image processing method) using the above-described ultrasonic measurement apparatus 1.

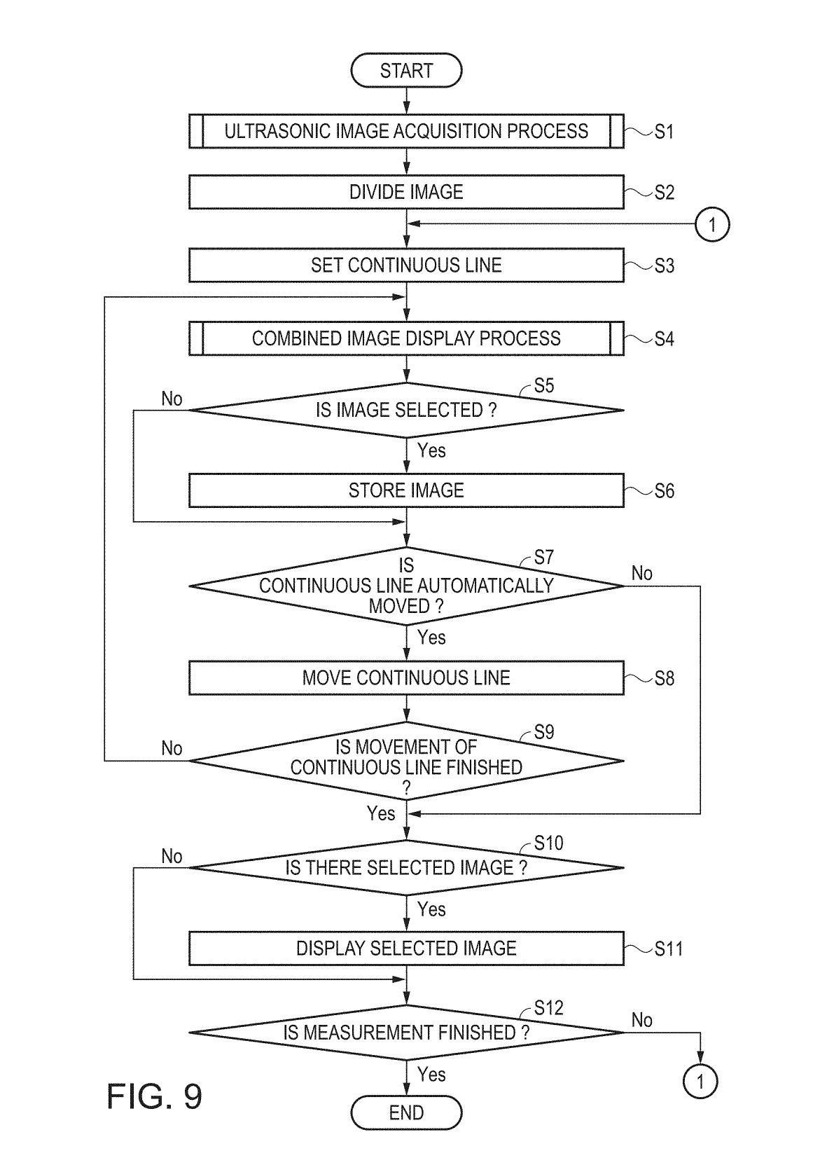

FIG. 8 is an image diagram illustrating a case where an ultrasonic measurement process is performed on the living body P by using the ultrasonic probe 2 of the present embodiment. FIG. 9 is a flowchart illustrating an ultrasonic measurement method in the present embodiment.

In the ultrasonic measurement method using the ultrasonic measurement apparatus 1 of the present embodiment, for example, an operator applies an acoustic matching agent (for example, a gel) for improving the propagation efficiency of an ultrasonic wave between the living body P and the ultrasonic sensor 22, on the sensor window 212A of the casing 21 of the ultrasonic probe 2. As illustrated in FIG. 8, the ultrasonic probe 2 is fixed to a skin surface of the living body P by using an adhesive tape or the like.

Acquisition of Internal Tomographic Image

Next, if an operation signal indicating that ultrasonic measurement is started by the operator operating the operation input unit, first, the control section 3 performs an ultrasonic image acquisition process (step S1; image acquisition step).

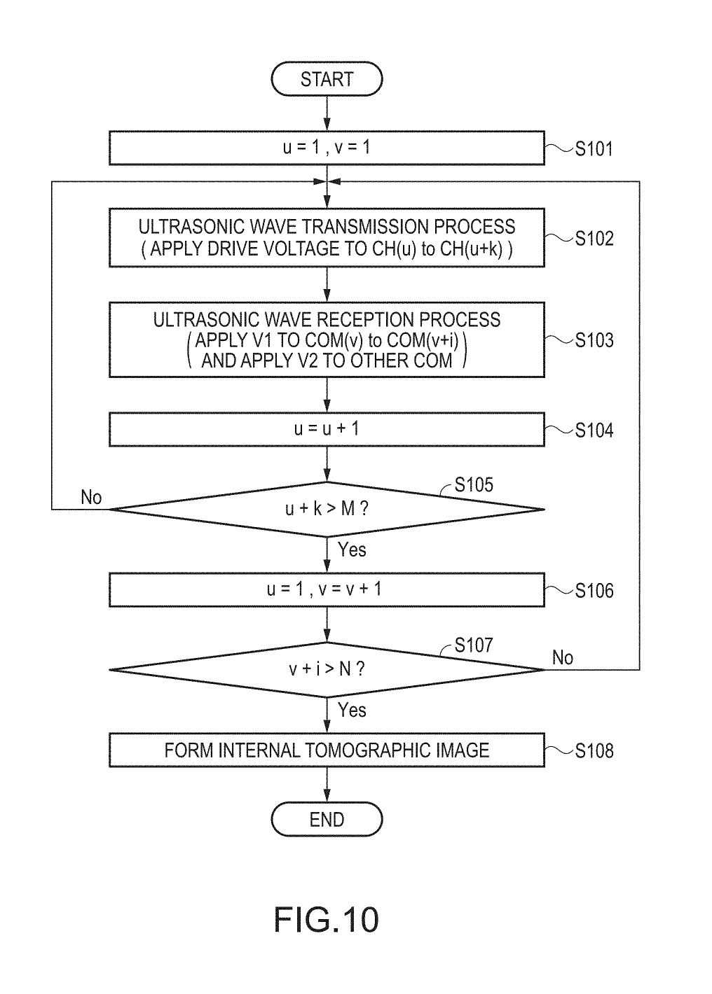

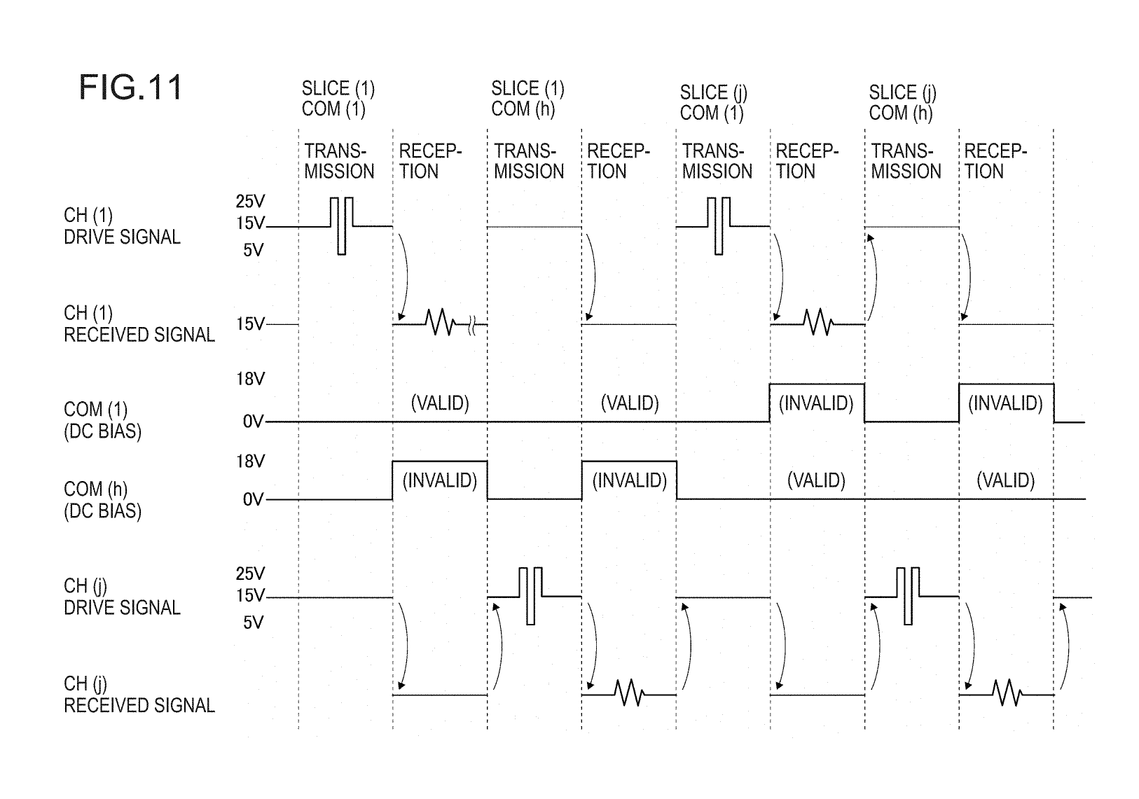

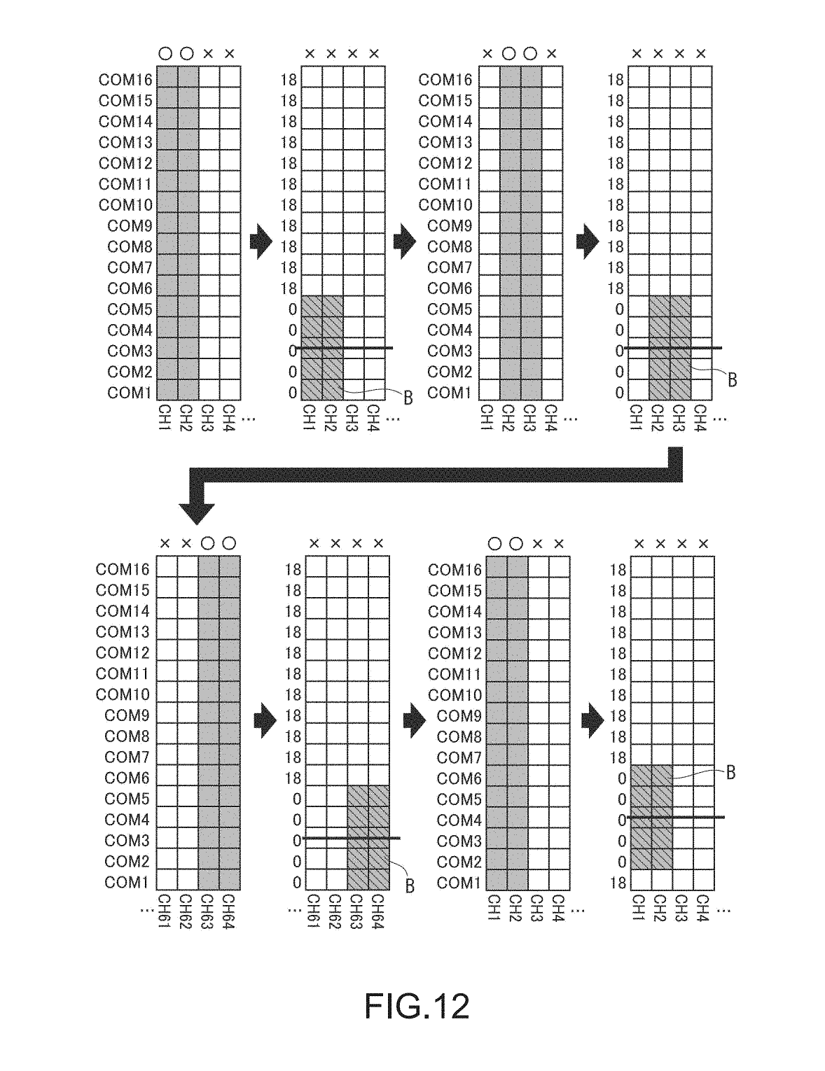

FIG. 10 is a flowchart illustrating the ultrasonic image acquisition process in step S1. FIG. 11 is a timing chart in the ultrasonic measurement process of the present embodiment. FIG. 12 is a diagram for explaining the order of driving the element portions 23 in the ultrasonic measurement process of the present embodiment.

In step S1, the control section 3 initializes a CH variable u and a COM variable v indicating a position of a driving target element portion 23 in ultrasonic measurement (u=1 and v=1) (step S101).

Here, the CH variable u (1≦u≦M) is a variable indicating a position (CH(1) to CH(M)) of the driving terminal 221D1 corresponding to the driving target element portion 23, and, in the present embodiment, a position (CH(1)) of the driving terminal 221D1 at an end on the −X side is set: to be u=1. In the present embodiment, M is 64. The COM variable v (1≦v≦N) is a variable indicating a position (COM(1) to COM(N)) of the common terminal 221E1 corresponding to the driving target (received signal acquisition target) element portion 23, and, in the present embodiment, a position (COM(1)) of the common terminal 221E1 at an end on the −Y side is set to be v=1. In the present embodiment, N is 16.

Next, the transmission control unit 31 performs an ultrasonic wave transmission process in which the element portions 23 corresponding to the CH variable u to the CH variable u+1 output ultrasonic waves (step S102).

Specifically, the transmission control unit 31 causes the switching circuit 253 to perform switching to transmission connection, and thus the transmission circuit 254 is connected to the respective driving terminals 221D1. As illustrated in FIG. 11, a predetermined voltage (for example, 15 V) is normally output to the driving terminals 221D1 regardless of a connection state of the switching circuit 253. In other words, a bias of +15 V is applied to the driving terminals 221D1. The transmission control unit 31 controls the second MUX 252 so that the first bias voltage V1 output from the voltage source 256 is output to all of the common terminals 221E1. For example, in a case where the bias voltage VB1 is 15 V, and a voltage of 15 V is output to the driving terminal 221D1, a voltage of which is the same as the first bias voltage V1 is output to all of the common terminals 221E1 during the ultrasonic wave transmission process.

The transmission control unit 31 outputs a pulsed drive signal from the transmission circuit 254. The transmission control unit 31 controls the first MUX 251 so that the drive signal is output to the driving terminals 221131 corresponding to the CH variable u to the CH variable u+k. Consequently, the pulse drive signal is output to the driving terminals 221D1 corresponding to positions of CH(u) to CH(u+k), and ultrasonic waves are transmitted from the respective element portions 23 (respective ultrasonic transducers 24) connected to the driving terminals 221D1. The integer k may be, for example, a preset value, and may be a value which is changed as appropriate by a user (an operator or the like). In the present embodiment, ultrasonic waves are output from a first number of (k+1) element portions 23 corresponding to CH(u) to CH(u+k) adjacent to each other in the X direction.

For example, in a case of the CH variable u=1, as illustrated in FIG. 11, a drive signal is output to the driving terminals 221D1 corresponding to a position of CH(1), and the drive signal is not output to the driving terminals 221D1 corresponding to CH(j) (where j is an integer of 3≦j≦64). In this case, as shown in a first state in FIG. 12, ultrasonic waves are transmitted from the element portions 23 corresponding to CH(1) and CH(2). In a case of the CH variable u=j, as illustrated in FIG. 11, a drive signal is output to the driving terminals 221D1 of CH (j) and the drive signal is not output to the driving terminals 221D1 of CH(1).

In a case of the integer k≧2, when an ultrasonic wave is transmitted, electronic focusing may be performed. In other words, in a plurality of driving terminals 221D1 to which a drive signal is output, an output timing of the drive signal is delayed from the end toward the center. Consequently, an ultrasonic wave which converges at a predetermined depth position, and a resolution in ultrasonic measurement can be improved.

Thereafter, the reception control unit 32 performs an ultrasonic wave reception process in which received signals are acquired from the element portions 23 corresponding to the COM variable v to the COM variable v+i (in the present embodiment, i=4) (step S103).

Specifically, the reception control unit 32 causes the switching circuit 253 to perform switching to reception connection so that the reception circuit 255 is connected to the respective driving terminals 221D1.

The reception control unit 32 controls the second MUX 252 so that the first bias voltage V1 output from the voltage source 256 is output to the respective common terminals 221E1 corresponding to the COM variable v to the COM variable v+i. The integer i may be, for example, a preset value, and may be a value which is changed as appropriate by a user (an operator or the like). In the present embodiment, the reception sensitivity of a second number of (i+l) element portions 23 corresponding to COM(v) to COM(v+i) adjacent to each other in the Y direction is greater than the reception sensitivity of other element portions 23, and thus received signals suitable for forming an internal tomographic image are acquired. Here, as described above, in the ultrasonic wave reception process, ultrasonic waves are transmitted from the element portions 23 corresponding to CH(u) to CH(u+k), but, among the element portions 23, the reception sensitivity of the element portions 23 corresponding to COM(v) to COM(v+i) increases. Therefore, reception in the ultrasonic wave reception process becomes valid in the element portions 23, and the reception sensitivity is nearly 0 in the other element portions 23, and thus reception becomes invalid. Only received signals from the element portions 23 in which reception is valid are input to the control section 3 via the reception circuit 255. Received signals from the element portions 23 at COM positions other than COM(v) to COM(v+i) are also input to the control section 3, but the second bias voltage is output thereto so that the reception sensitivity thereof decreases. Thus, the received signals are also reduced to the extent of not influencing measurement thereof.

In other words, a region (+Z side) directly under the element portions 23 (ultrasonic transducers 24) which are connected to the driving terminals 221D1 corresponding to CH (u) to CH(u+k) and are connected to the common terminals 221E1 corresponding to COM(v) to COM(v+i) is an ultrasonic measurement target region (measurement region B (refer to FIG. 12)) in the first step S102 and step S103.

As a specific example, for example, in a case where the COM variable v is 1, as illustrated in FIG. 11, during the ultrasonic wave reception process, the first bias voltage V1 which causes reception to be valid in the element portions 23 (ultrasonic transducers 24) is output to the common terminal 221E1 at the position corresponding to COM 1).

On the other hand, during the ultrasonic wave reception process, second bias voltage V2 which causes reception to be invalid in the element portions 23 (ultrasonic transducers 24) is output to the common terminal 221E1 corresponding to COM(h) (where h is an integer of 5≦h≦16). In a case where a bias voltage which causes reception to be invalid is −3 V, a voltage of 18 V is output as the second bias voltage V2.

Here, in a case where the CH variable u is 1, as shown in a second state in FIG. 12, received signals indicating that ultrasonic waves are detected are output from the respective element portions 23 corresponding to the measurement region B in which ultrasonic wave transmission positions overlap positions where reception of ultrasonic waves is valid. Therefore, as illustrated in FIG. 11, a received signal having a high level due to reception of an ultrasonic wave is output from the driving terminal 221D1 corresponding to CH(1), and a level of a received signal from the driving terminal 221D1 corresponding to CH(j) is less than a predetermined value. On the other hand, in a case where the CH variable u is j, a received signal having a high level due to reception of an ultrasonic wave is output from the driving terminal 221D1 corresponding to CH(j), and a level of a received signal from the driving terminal 221D1 corresponding to CH(1) is less than a predetermined value.

Similarly, in a case where the COM variable v is h, a received signal having a high level due to reception of an ultrasonic wave is output from the element portions 23 (the element portions 23 corresponding to the measurement region B) in which positions for outputting ultrasonic waves and positions for outputting the first bias voltage V1 overlap each other.

Thereafter, a predetermined value (for example, “1”) is added to the CH variable u (step S104), and it is determined whether or not the CH variable u+k exceeds the maximum value M (in the present embodiment, M=64) of the element portions 23 arranged in the X direction (step S105).

In a case where a determination result is negative (No) in step S105, the flow returns to step S102. In other words, as shown in third and fourth states in FIG. 12, a CH position for transmitting an ultrasonic wave is moved to the +X side, and a scanning process in the X direction, for acquiring received signals from COM(v) to COM(v+i) is continuously performed. On the other hand, in a case where a determination result is affirmative (Yes) in step S105, as shown in fifth and sixth states in FIG. 12, this indicates that a CH position for transmitting an ultrasonic wave reaches the end on the +X side (one scanning process is finished). In other words, received signals required for internal tomographic images (internal tomographic images corresponding to y=1) along the X direction corresponding to the positions of COM(1) to COM(5) are obtained.

In this case, the next section position of the living body P starts to be measured. For this, the control section 3 initializes the CH variable u (u=1), and adds a predetermined value (for example, “1”) to the COM variable v (step S106). It is determined whether or not the COM variable v+i exceeds the maximum value N (in the present embodiment, N=16) of the element portions 23 arranged in the Y direction (step S107). In a case where a determination result is negative (No) in step S107, the flow returns to step S102. In other words, as shown in seventh and eighth states in FIG. 12, a COM position for acquiring a received signal is moved to the +Y side, and received signals are sequentially acquired from COM(v) to COM(v+i). Thereafter, a scanning process in the X direction is performed in a plurality until a determination result is affirmative (Yes) in step S107.

On the other hand, in a case where a determination result is affirmative (Yes) in step S107, this indicates that transmission and reception processes of ultrasonic waves for all of the element portions 23 of the array region 22A are completed.

In this case, the image acquisition unit 33 forms (acquires) an internal tomographic image on the basis of ultrasonic measurement results (step S108). In other words, the image acquisition unit 33 generates ultrasonic wave reflection positions based on ultrasonic wave transmission timings and reception timings as images, so as to generate an internal tomographic image along the X direction corresponding to COM(v) to COM(v+i). In the present embodiment, received signals for a single internal tomographic image are acquired through a scanning process in the X direction, and the scanning process is sequentially performed while a position is deviated in the Y direction, Therefore, internal tomographic images at a plurality of positions in the Y direction can be acquired.

Display of Combined Tomographic Image

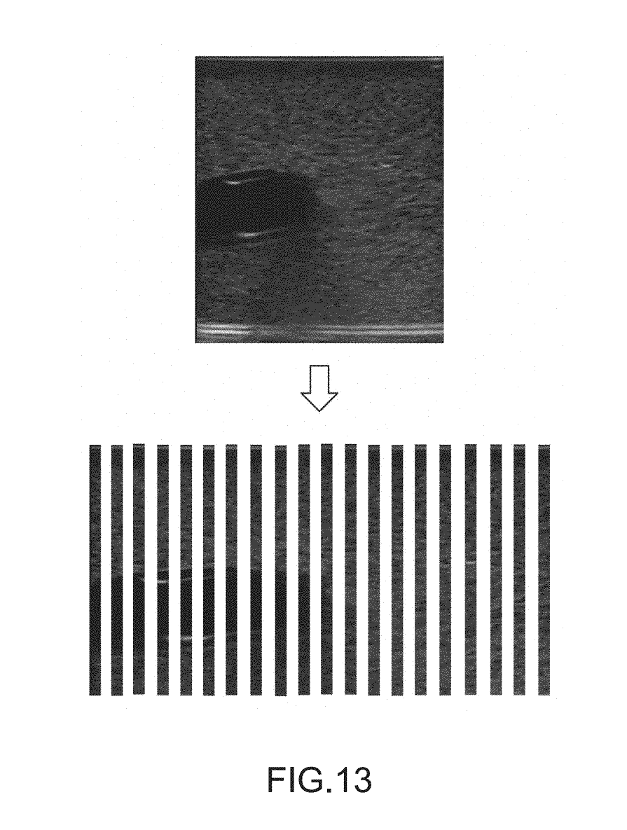

Referring to FIG. 9 again, after the above-described ultrasonic image acquisition process in step S1, the image dividing unit. 34 divides each acquired internal tomographic image into a plurality of separate images (step S2; image dividing step). FIG. 13 is a diagram illustrating an example of generating separate images from an internal tomographic image.

In step S2, as illustrated in FIG. 13, the image dividing unit 34 generates X_{M} separate images (image size: 1×Z_{M}) corresponding to respective pixels (1≦x≦Xₘ) in the X direction with respect to a single internal tomographic image, Since Y_{M} internal tomographic images are acquired in the Y direction, X_{M}×Y_{M} separate images are generated.

Next, the control section 3 sets a continuous line indicating positions where images of an internal tomographic structure are displayed on the XY plane (step S3). In step S3, it is possible to set whether a continuous line is manually set on the basis of an operation signal from an operator via the operation input unit, or a continuous line is automatically moved. For example, in a case where the operator manually inputs an operation signal indicating a position of a continuous line, a manual mode is determined, and an input continuous line is set. In a case where an operation signal indicating a position of a continuous line is not input, an automatic mode is determined, In the automatic mode, continuous line which is set in advance is initially set, and scanning is performed by automatically moving the continuous line in a rectangular region on the XY plane.

The rectangular region mentioned here is a region substantially corresponding to the array region 22A, and is a region of 1≦x≦X_{M} and 1≦y≦Y_{M}Since a total number of driving terminals 221D1 is M, a total number of common terminals 221E1 is N, a width (the number of CH) of the measurement region B in the X direction is k+1, and a width (the number of COM) thereof in the Y direction is j+1, X_{M} is M−k, and Y_{M} is N−j. In the present embodiment, since M is 64, N is 16, k is 1, and j is 4, x and y are in ranges of and 1≦x≦63, and 1≦y≦12.

Therefore, a corresponding separate image (s, t) is present at a coordinate (s, t) in the rectangular region.

In the following description, four vertices of the rectangular region are respectively defined as a first vertex (1, Y_{M}), a second vertex (X_{M}, Y_{M}) , a third vertex (1, Y_{M}) and a fourth vertex (1,1) clockwise.

In a case where the automatic mode is determined in step S3, the control section 3 sets an initial continuous line as a straight line passing through the first vertex and the second vertex.

Thereafter, the control section 3 performs a combined image display process of generating and displaying a combined tomographic image corresponding to the continuous line (step S4; image combining step).

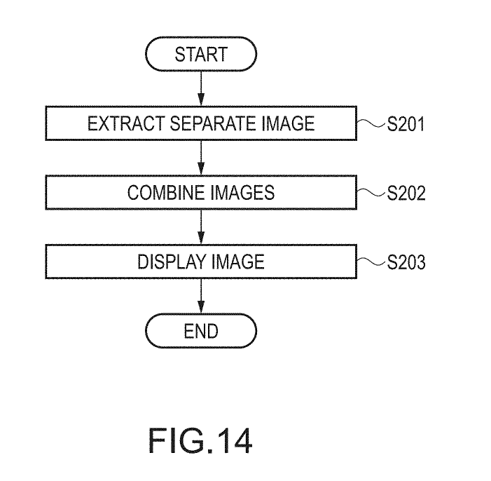

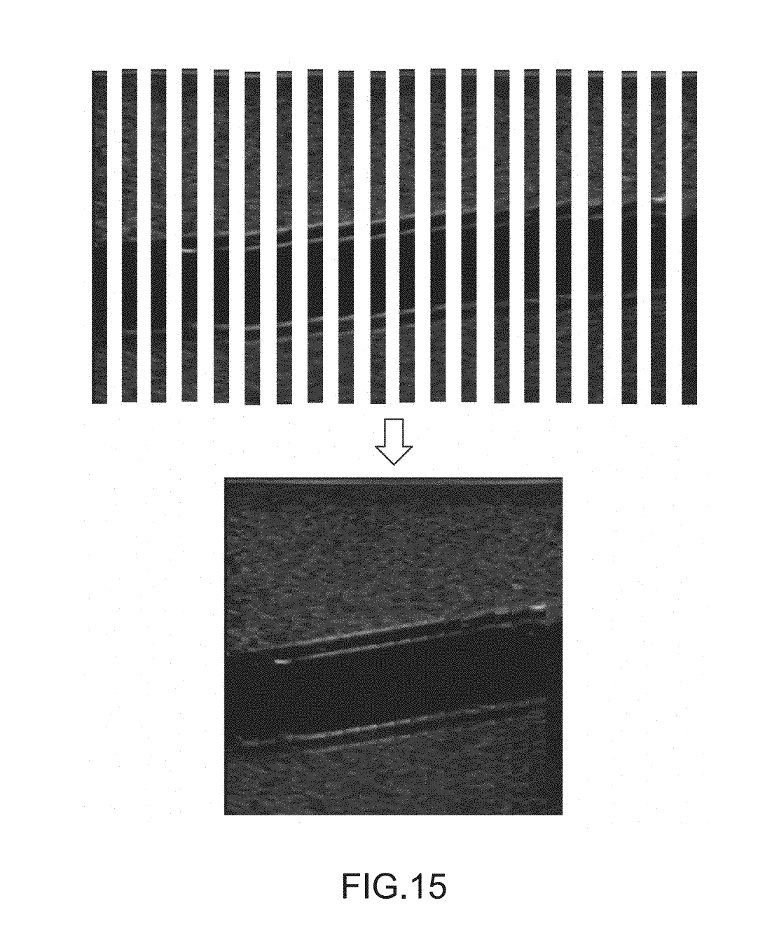

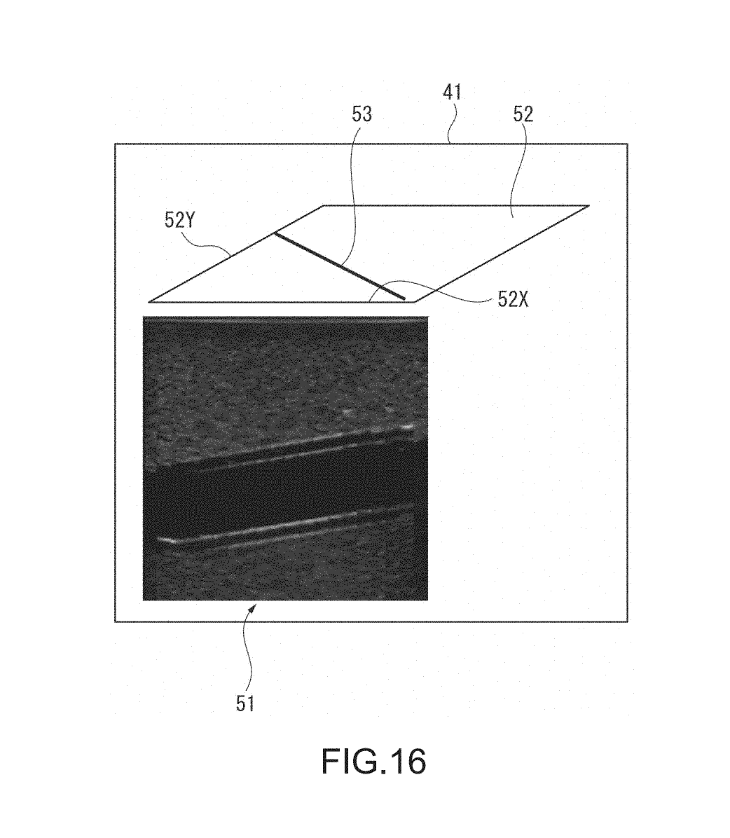

FIG. 14 is a flowchart illustrating the combined image display process in step S4. FIG. 15 is a diagram illustrating an example of generating a combined tomographic image. FIG. 16 is a diagram illustrating a combined tomographic image displayed on the display section.

In the combined image display process, as illustrated in FIG. 13, the image combining unit 35 extracts separate images corresponding to coordinates on the continuous line from among the separate images obtained through division instep S2 (step S201). In otherwords, a linear expression y=f(x) for the continuous line is calculated, t=f(1) to f(X_{M}) corresponding to s=1 to X_{M} is calculated, and a separate image (s, t) is extracted. In a case where the Y coordinate value t corresponding to the X coordinate value s of the linear expression for the continuous line is not an integer, the closest integer value may be used as the value t.

Next, the image combining unit 35 disposes a separate image (1,f(1)) at an end on a −S side in a coordinate system (a transverse axis is set to an S axis, and a longitudinal direction is set to a Z axis) of the combined image, disposes separate images t) corresponding to s=2, 3, . . . , and X_{M} in order toward a S side, and combines the separate images with each other so as to generate a combined tomographic image (step S202). Consequently, as illustrated in FIG. 15, the combined tomographic image is generated on the basis of the separate images.

Next, the display control unit 38 displays the combined tomographic image generated in step S202 on the display section 4 (step S203).

Here, as illustrated in FIG. 16, the display control unit 38 displays a combined tomographic image 51 (or an internal tomographic image in a case where the continuous line is parallel to the X direction), a simple array image 52 corresponding to the array region 22A which corresponds to the combined tomographic image 51, and a section position image 53 indicating a position of the continuous line with respect to the array region 22A in an arranged manner in a display region 41 of the display section 4.

At this time, the display control unit 38 displays an X axis image 52X and a Y axis image 52Y on the simple array image 52, and then displays the section position image 53 to overlap the simple array image 52. Consequently, it is possible to allow an operator to easily understand a section position of the displayed combined tomographic image 51 (internal tomographic image).

Referring to FIG. 9 again, after the above-described combined image display process, the image selecting unit 36 determines whether or not an operation signal indicating that the operator selects the displayed combined tomographic image 51 is input (step S5). In a case where a determination result is affirmative (Yes) in step S5, the image selecting unit 36 stores the combined tomographic image 51 at a timing at which the operation signal is input, in the storage unit (step S6).

In other words, in the present embodiment, in a case where the continuous line is automatically moved in the automatic mode, a combined tomographic image obtained when the continuous line is moved is displayed on the display section 4 in real time (displayed in an animation manner). Therefore, in a case where the combined tomographic image 51 (internal tomographic image) at a section position desired to be observed is displayed, the operator operates an operation unit (for example, clicks on a mouse) so as to select the image. Even in the manual mode, since an image can be selected by the image selecting unit 36, it is possible to save time and effort to set a continuous line again, for example, in a case where a combined tomographic image at the same position is desired to bye observed.

After step S6, and after a determination result is negative (No) in step S5, the image combining unit 35 determines whether or not the continuous line is moved (step S7). In step S7, it is determined whether or not the automatic mode is set in a state in which the continuous line is not manually input in the above step S3.

In a case where a determination result is affirmative (Yes) in step S7, the image combining unit 35 of the control section 3 moves the continuous line to a preset direction (step S8). In the present embodiment, in step S8, the continuous line is moved as follows.

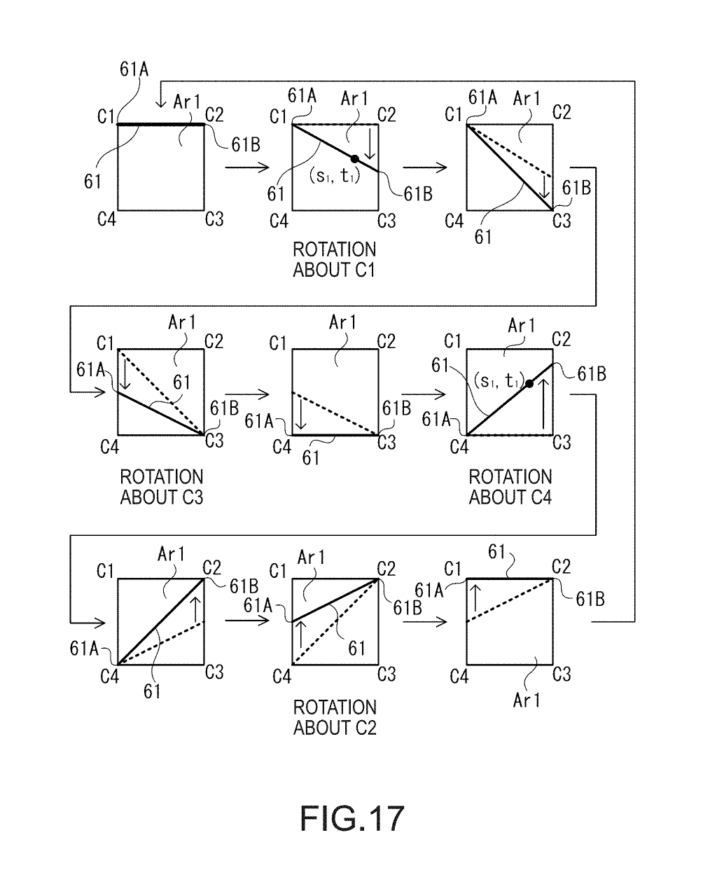

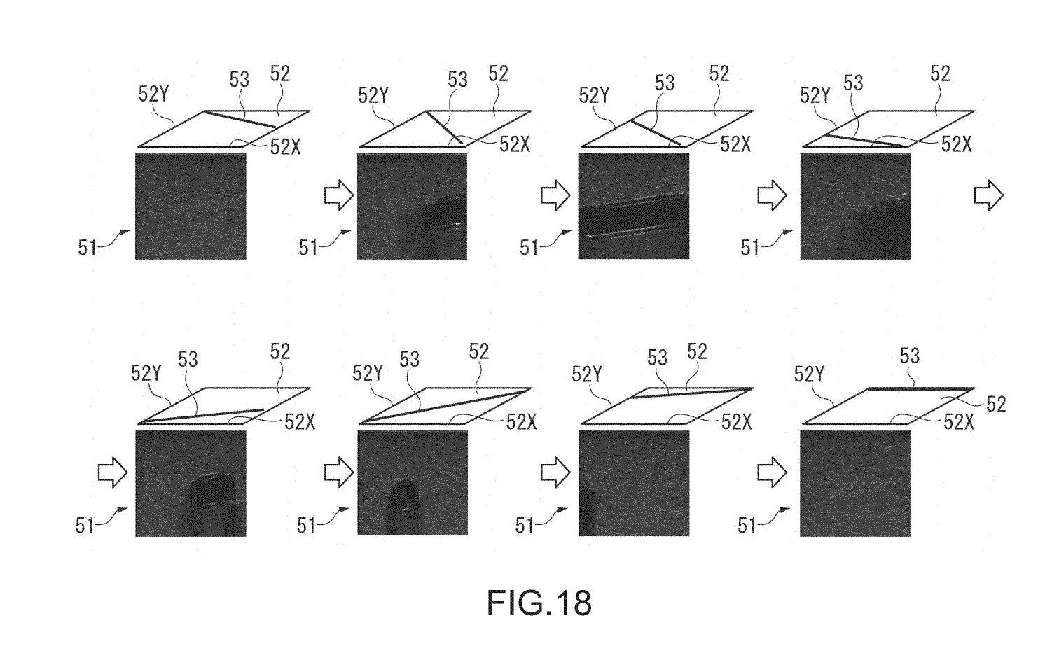

FIG. 17 is a diagram for explaining movement procedures of the continuous line in the present embodiment FIG. 18 is a diagram illustrating an example of transition of the combined tomographic image 51 displayed on the display section 4 in the present embodiment.

In a case where the automatic mode is set, in step S3, as illustrated in FIG. 17, an initial continuous line 61 is set to a straight line passing through a first vertex C1 and a second vertex C2, In other words, the continuous line 61 corresponding to a section position of an internal tomographic image acquired last in step S1 is set. Here, among intersections between the continuous line 61 and an outer peripheral edge of a rectangular region Ar1, a point overlapping the first vertex is referred to as a first intersection 61A, and a point overlapping the second vertex is referred to as a second intersection 61B.

In the present embodiment, the image combining unit 35 first moves the second intersection 61B toward a third vertex C3 with the first intersection 61A in the above-described initial continuous line 61 as the rotation center (first point), so as to rotate the continuous line 61.

If the second intersection 61B is moved to the third vertex C3, next, the rotation center is moved to the second intersection 61B, and the first intersection 61A is moved from the first vertex C1 toward a fourth vertex C4, so that the

continuous line 61 is rotated. If the first intersection 61A is moved to the fourth vertex C4, next, the rotation center is moved to the first intersection 61A, and the second intersection 61B is moved from the third vertex C3 toward the second vertex C2, so that the continuous line 61 is rotated.

If the second intersection 61B is moved to the second vertex C2, the rotation center is moved to the second intersection 61B, the first intersection 61A is moved from the fourth vertex C4 toward the first vertex C1 so that the continuous line 61 is rotated, and thus the continuous line 61 is returned to the initial position.

In the present embodiment as mentioned above, end points (the first intersection 61A and the second intersection 61B) of the continuous line 61 are alternately replaced with each other, and the continuous line 61 is rotated with each of the four vertices of the rectangular region Ar1 as the rotation center. In this case, as illustrated in FIG. 17, each position in the rectangular region Ar1 is scanned at least twice so that a direction of the continuous line 61 differs. For example, with respect to a predetermined coordinate (s₁, t₁) in the rectangular region Ar1, a combined tomographic image corresponding to a case where an inclination of the continuous line 61 is negative (the right-downward continuous line 61 shown on the second part in FIG. 17), and a combined tomographic image corresponding to a case where an inclination of the continuous line 61 is positive (the right-upward continuous line 61 shown on the sixth part in FIG. 17) can be obtained. Therefore, in a case where a line direction (long axis direction) of a blood vessel is directed from the upper left toward the lower right, the operator can appropriately determine the long axis direction of the blood vessel on the basis of the former combined tomographic image, and, in a case where the long axis direction of the blood vessel is directed from the lower left toward the upper right, the operator can appropriately determine the long axis direction of the blood vessel on the basis of the latter combined tomographic image. Meanwhile, an amount of moving the continuous line 61 is a preset amount, and may be, for example, an amount corresponding to one coordinate in the rectangular region, and may be an amount corresponding to two or more coordinates. After the continuous line 61 is moved by a predetermined amount, the control section 3 returns to the process in step S4 unless a determination result is affirmative (Yes) in step S9 which will be described later. In other words, the processes in steps S4 to S7 are repeatedly performed while moving the continuous line 61 until a determination result is affirmative (Yes) in step S9. Consequently, as illustrated in FIG. 18, combined tomographic images corresponding to movement destinations of the continuous line 61 are sequentially displayed on the display section 4, and the combined tomographic image 51 (or an internal tomographic image) corresponding to movement of the continuous line 61 is displayed in real time (displayed in an animation manner).

Referring to FIG. 9 again, after the above step S8, the control section 3 determines whether or not movement of the continuous line 61 is finished (step S9). In step S9, in a case where an operation signal indicating that the operator finishes real-time display of the combined tomographic image 51, or combined tomographic images are displayed in real time through movement of the continuous line 61 for a predetermined number of times (or a predetermined time) the control section 3 determines that movement of the continuous line 61 is finished in step S9. In a case where it is determined that movement of the continuous line 61 is not finished in step S9, the flow returns to the process in step S4.

In a case where a determination result is affirmative (Yes) in step S9, the display control unit 38 determines whether or riot there is a selected image stored in step S6 (step S10). In a case where a determination result is affirmative (Yes) in step S10, the display control unit 38 displays the selected combined tomographic image 51 (or the internal tomographic image) stored in the storage unit on the display section 4. In a case where there are a plurality of combined tomographic images 51, the plurality of combined tomographic images 51 may be sequentially displayed in a switching manner, and may be displayed in a list form in the display region 41 of the display section 4.

In a case where a determination result is negative (No) in step S10, and after step S11, the control section 3 determines whether or not the measurement process is finished (step S12). For example, in step S12, in a case where an operation signal indicating that the operator finishes the measurement through an input operation is input, the measurement process is finished. In a case where a determination result is negative (No) in step S12, the flow returns to step S3, Consequently, the operator can display the combined tomographic image 51 in the automatic mode, and can then display the combined tomographic image 51 by designating the continuous line 61 again in the manual mode. After the manual mode, in a case where a position of a blood vessel cannot be favorably determined, the combined tomographic image 51 can be displayed in real time through switching to the automatic mode.

Here, a description has been made of an example in which the flow returns to step S3 in a case where a determination result is negative (No) in step S12, but the flow may return to step Si. In ultrasonic measurement targeting the living body P, a position of a blood vessel changes over time. In this case, the flow returns to step Si so that an internal tomographic image is acquired again, and thus it is possible to perform measurement with higher accuracy.

Display of Combined Tomographic Image obtained by Changing Rotation Center

Meanwhile, in the above-described example, in the automatic mode, a description has been made of an example in which, when the end point (the first intersection 61A or the second intersection 61B) of the continuous line 61 is located at each vertex of the rectangular region Ar1, the combined tomographic image Si obtained by rotating the continuous line 61 about the vertex is displayed.

In contrast, in the present embodiment, after the ultrasonic measurement process (first ultrasonic measurement process) as illustrated in FIG. 9, a combined tomographic image obtained when the operator rotates the continuous line 61 centering on any point may be displayed (second ultrasonic measurement process).

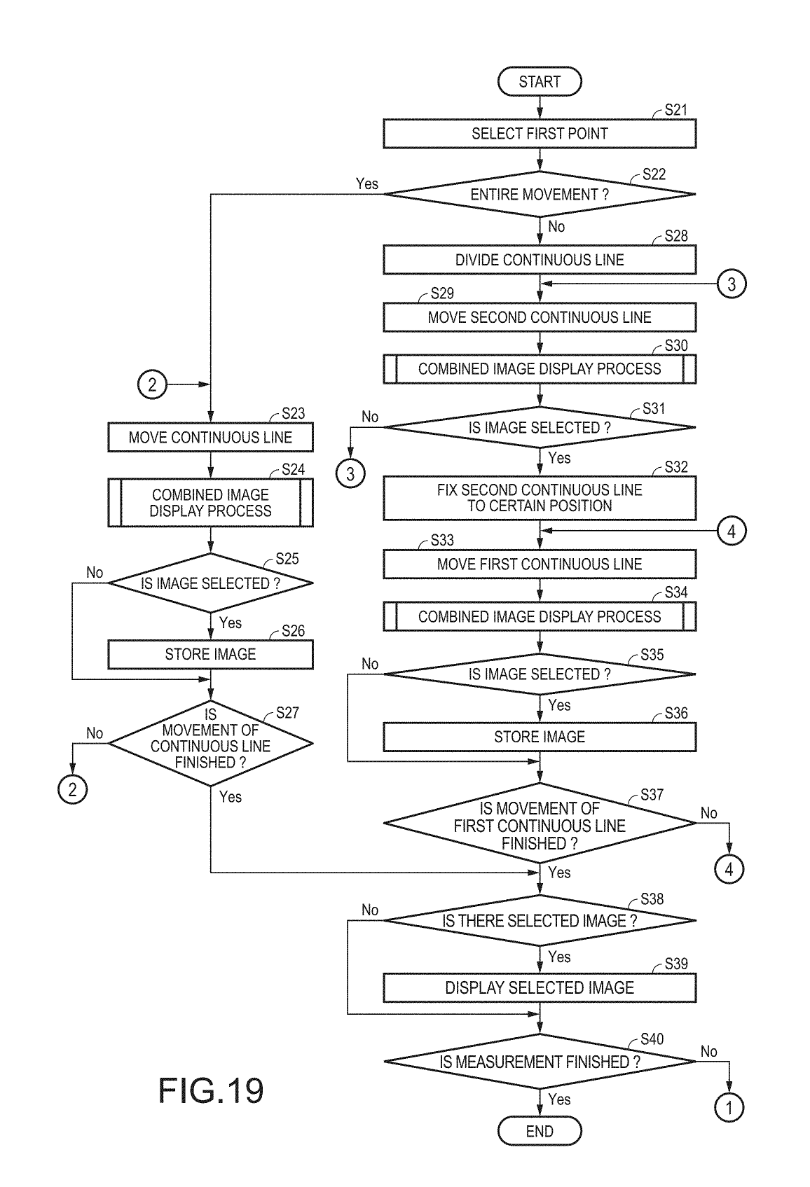

FIG. 19 is a flowchart illustrating the second ultrasonic measurement process.

In the second ultrasonic measurement process, for example, in the above-described first ultrasonic measurement process, a predetermined combined tomographic image 51 is selected and is displayed on the display section 4, and then the first point selecting unit 37 acquires a first point. In other words, in a case where the operator performs an input operation on the operation input unit, and an operation signal indicating that a certain point on a combined tomographic image displayed on the display section 4 is designated as a first point is input, the first point selecting unit 37 acquires the input point as a first point (a point serving as the rotation center) (step S21). The first point is a point on the selected combined tomographic image 51, and is a point indicating a separate image (s, t) forming the combined tomographic image 51, that is, a point located on the continuous line 61. For example, in a coordinate system (a transverse axis is set to an S axis, and a longitudinal direction is set to a Z axis) of the combined image, in a case where any point (s, z) on the combined tomographic image 51 is designated, the first point selecting unit 37 determines that a separate image (s,) including the point (s, z), that is, the point (s ,t) on the continuous line 61 is selected.

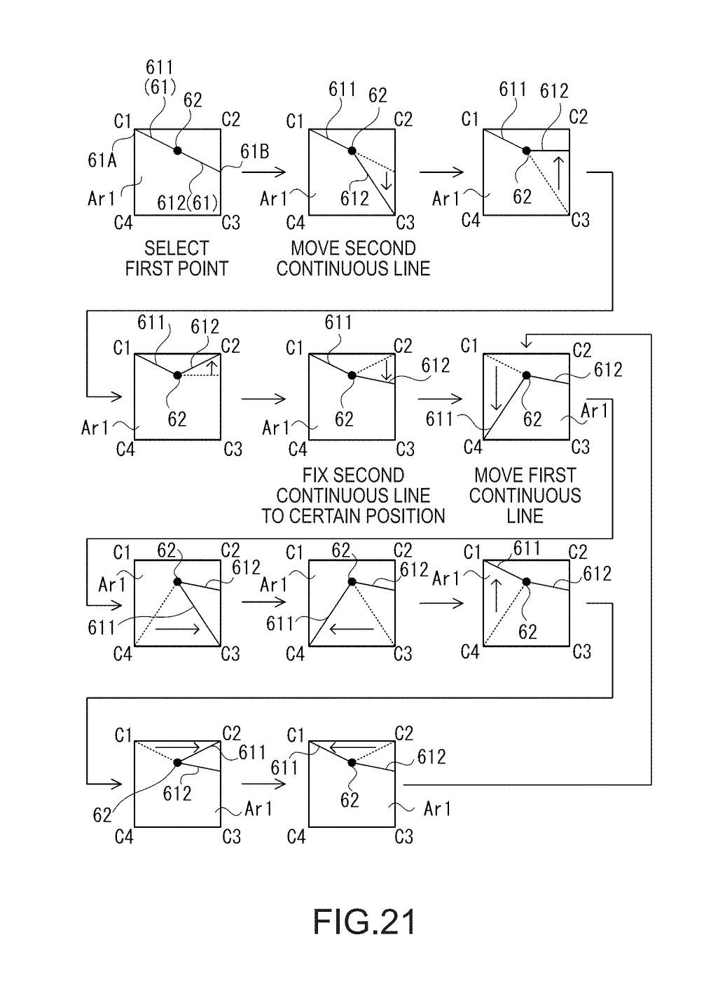

Next, the first point selecting unit 37 determines whether or not the entire movement is selected (step S22). Here, the entire movement indicates rotation scanning in which the entire continuous line 61 is rotated centering on the first point. In a case where the entire movement is not selected, in the present embodiment, partial movement is performed. The partial movement indicates rotation scanning in which, when the continuous line 61 i divided into two straight lines (a first continuous line 611 (refer to FIG. 21) and a second continuous line 612 (refer to FIG. 21)) with the point (s, t) on the continuous line 61 interposed therebetween, the first continuous line 611 and the second continuous line 612 are separately rotated. It is assume& that the first continuous line 611 is a line segment located further toward the first intersection 61A side than the first point, and the second continuous line 612 is a line segment located further toward the second intersection 61B side than the first point.

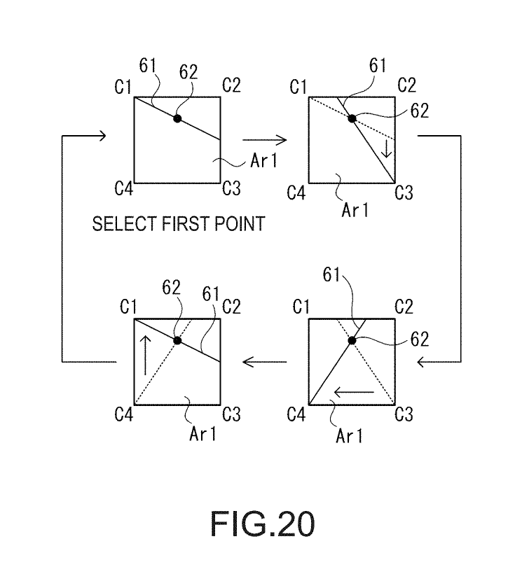

In a case where an operation signal indicating that the entire movement is designated is input by the operator operating the operation input unit, the first point selecting unit 37 determines that the entire movement is selected in step S22, FIG. 20 is a diagram for explaining movement procedures when the entire continuous line 61 is rotated centering on a first point 62.

In a case where a determination result is affirmative (Yes) in step S22, as illustrated in FIG. 20, the image combining unit 35 rotates the continuous line 61 with the first point 62 selected in step S21 as the rotation center (step S23). A rotation angle is a preset angle in the same manner as in the movement of the continuous line 61 in step S8. The image combining unit 35 performs the same combined image display process as in step S4 so as to display the combined tomographic image 51 (step S24). At this time, in the same manner as in steps S5 and S6, the image selecting unit 36 determines whether or not an operation signal indicating that the operator selects the combined tomographic image 51 is input (step S25). If the combined tomographic image 51 is selected, the combined tomographic image 51 is stored in the storage unit (step S26). The control section 3 determines whether or not movement of the continuous line 61 is finished (step S27). In the same manner as in step S9, in step S27, for example, in a case where an operation signal indicating that the operator finishes real-time display of the combined tomographic image 51, or combined tomographic images 51 are displayed in real time through movement of the continuous line 61 for a predetermined angle (or a predetermined time), the control section 3 determines that movement of the continuous line 61 is finished. In a case where it is determined that movement of the continuous line 61 is not finished in step S27, the flow returns to the process in step S23, and the combined tomographic image 51 obtained when the continuous line 61 is rotated is continuously displayed in real time. Therefore, in step S27, the continuous line 61 is rotated centering on the first point until a determination result is affirmative (Yes) in step S27, and the combined tomographic image 51 generated at that time is displayed on the display section 4 in real time.

FIG. 21 is a diagram for explaining movement procedures of the continuous line 61 in a case where the continuous line 61 is divided with the first point 62 as a boundary.

In a case where a determination result is negative (No) in the above step S22, the image combining unit 35 divides the continuous line 61 into the first continuous line 611 on the first intersection 61A side and the second continuous line 612 on the second intersection 61B with the first point 62 as a boundary (step S28). The image combining unit 35 rotates the second continuous line 612 centering on the first point 62 (step S29).

Specifically, as illustrated in FIG. 21, the image combining unit 35 first rotates the second continuous line 612 in a clockwise direction, and then rotates the second continuous line 612 in a counterclockwise direction. For example, in a case where the second intersection 61B is located between the second vertex C2 and the third vertex C3, the second continuous line 612 is first rotated until the second intersection 61B is located at the second vertex C2, then, the rotation direction is inverted, and the second continuous line 612 is rotated until the second intersection 61B is located at the third vertex C3.

A rotation angle at this time is a preset angle in the same manner as in step S8 or step S23. In the same mariner as in step S4, the image combining unit 35 performs a combined image display process whenever the second continuous line 612 is rotated by a predetermined angle, so as to display the combined tomographic image 51 on the display section 4 in real time (step S30).

In the same manner as in step S5, the image selecting unit 36 determines whether or not an operation signal indicating that the operator elects the combined tomographic image 51 is input (step S31).

In a case where a determination result is negative (No) in step S31, the flow returns to step S29, and movement of the second continuous line 612 is continuously moved.

On the other hand, in a case where a determination result is affirmative (Yes) in step S31, the image combining unit 35 stops movement of the second continuous line 612, and fixes the second continuous line 612 to a certain position (step S32)

After step S32, the image combining unit 35 moves the first continuous line 611 (step S33).

Specifically, as illustrated in FIG. 21, the image combining unit 35 first rotates the first continuous line 611, for example, in a counterclockwise direction, and then rotates the first continuous line 611 in a clockwise direction. For example, in a case where the first intersection 61A is located at the first vertex C1, first, the first continuous line 611 is rotated until the first intersection 61A is moved to the third vertex 03 via the fourth vertex 04, then, the rotation direction is inverted, and the first continuous line 611 is rotated until the first intersection 61A is moved to the second vertex 02 via the third vertex C3 and the first vertex C1.

A rotation angle at this time is a preset angle in the same manner as in step S8, step S23, or step S29. In the same manner as in step S4, the image combining unit 35 performs a combined image display process so as to display the combined tomographic image 51 on the display section 4 in real time (step S34) in the same manner as in steps S5 and S6, the image selecting unit 36 determines whether or not an operation signal indicating that the operator selects the combined tomographic image is input (step S35), and stores the combined tomographic image 51 in the storage unit in a case where a determination result is affirmative (Yes) (step S16).

The control section 3 determines whether or not movement of the first continuous line 611 is finished (step S37). In the same manner as in step S9, in step S37, for example, in a case where an operation signal indicating that the operator finishes real-time display of the combined tomographic image 51, or the combined tomographic image 51 is displayed in real time through movement of the continuous line 61 for a predetermined angle (or a predetermined time), the control section 3 determines that movement of the continuous line 61 is finished, In a case where a determination result is negative (No) in step S37, the flow returns to step S33.

In a case where a determination result is affirmative (Yes) in step S37, the same processes (from step S38 to step S40) as the processes in steps S10 to S12 in FIG. 9 are performed.

Operations and Effects of Present Embodiment

In the ultrasonic measurement apparatus 1 of the present embodiment, the image acquisition unit 33 acquires a plurality of internal tomographic images along the Y direction, and the image dividing unit 34 divides each internal tomographic image into a plurality of separate images with a normal line to the X direction. The image combining unit 35 extracts a separate image (s, t) corresponding to each coordinate (s, t) on the continuous line 61 which is continued on the XY plane which is the same plane as the array region 22A, arranges the extracted separate images in order of coordinates along the continuous line 61 so as to combine the separate images with each other, and thus generates a combined tomographic image. Thus, as illustrated in FIG. 8, an operator can check the combined tomographic image 51 indicating a sectional structure of the living body P corresponding to a desired position of the continuous line without changing a position or an angle of the ultrasonic probe 2 in a state in which the ultrasonic probe is fixed to the living body P. Consequently, for example, when puncture work is performed, a line direction of a blood vessel in the living body P can be easily recognized, and thus the operator can also easily understand an insertion direction of inserting the puncture needle 11. Therefore, it is possible to efficiently perform puncture work and to improve a puncturing success ratio.

In the ultrasonic measurement apparatus 1 of the present embodiment, the continuous line 61 is a straight line. Thus, when the linear puncture needle 11 is inserted into a blood vessel, a line direction (long axis direction) of the blood vessel can be specified by checking the combined tomographic image 51. In other words, an operator can easily understand an insertion direction in which the puncture needle 11 is easily inserted by checking the combined tomographic image 51 in which the maximum dimension of the blood vessel in the long axis direction is greatest and a position of the continuous line 61 at that time.

In the ultrasonic measurement apparatus 1 of the present embodiment, the display control unit 38 displays the combined tomographic image 51 obtained when the continuous line 61 is moved, on the display section 4 in real time. In a case where an input operation for selecting an image by using the image selecting unit 36 is performed, the display control unit 38 displays the combined tomographic image 51 at a timing of performing the input operation. Consequently, the operator can easily check a sectional structure of the living body P at various positions when the continuous line 61 is moved. Since the combined tomographic image 51 corresponding to a desired continuous line 61 at a timing designated by the operator can be displayed, the operator can easily check the desired combined tomographic image 51.

In the ultrasonic measurement apparatus 1 of the present embodiment, the display control unit 38 displays the combined tomographic image 51 on the display section 4, and also displays a position of the continuous line 61 corresponding to the combined tomographic image 51 by using the simple array image 52 and the section position image 53.

Consequently, the operator can easily understand a position in the living body P corresponding to the combined tomographic image 51 displayed on the display section 4.

In the ultrasonic measurement apparatus 1 of the present embodiment, the image combining unit 35 generates the combined tomographic image 51 obtained when the continuous line 61 passing through the first point 62 is rotated centering on the first point 62, and displays the combined tomographic image 51 in real time.

Consequently, internal tomographic images passing through respective coordinate positions in the rectangular region Ar1 can be displayed. Since the first point 62 can be set and input by the operator, for example, if a point on a blood vessel is set as the first point 62, a combined tomographic image in which a line direction of the blood vessel is reflected and a position of the continuous line 61 at that time can be easily acquired. Therefore, it is possible to efficiently perform puncture work and to improve a puncturing success ratio.

In the ultrasonic measurement apparatus 1 of the present embodiment, the image combining unit 35 rotates the continuous line 61 by using the vertex of the rectangular region Ar1 as the first point (rotation center).

In a case of generating a combined tomographic image obtained when the continuous line 61 is rotated by using a vertex position of the rectangular region Ar1 as the rotation center, combined tomographic images in various directions can be obtained by appropriately changing a vertex used as the rotation center, and thus the operator more accurately determines a line direction of a blood vessel.

In the ultrasonic measurement apparatus 1 of the present embodiment, the image combining unit 35 generates a combined tomographic image obtained when the continuous line 61 is rotated by using each vertex of the rectangular region Ar1 as the rotation center.

In this case, since each combined tomographic image can be acquired when the continuous line is rotated with each vertex as the center, even if a line direction of a blood vessel is any direction in the rectangular region Ar1, a continuous line close to the line direction of the blood vessel can be easily detected. After scanning centering on the vertex of the rectangular region Ar1 is performed in the automatic mode, and a mark in a line direction of a blood vessel is attached, scanning in which the first point 62 is designated is performed, or a combined tomographic image is displayed in the manual mode in a state in which a continuous line is designated as illustrated in FIG. 19, and thus the operator can detect the line direction of the blood vessel with higher accuracy.

In the present embodiment, the image combining unit 35 rotates the continuous line 61 with the first intersection 61A of the continuous line 61 as the rotation center, and then rotates the continuous line 61 with the second intersection 61B as the rotation center. In other words, the rotation center during rotation of the continuous line 61 alternately switches between the first intersection 61A and the second intersection 61B. Consequently, when each generated combined tomographic image is displayed in real time (displayed in an animation manner), a horizontal position of an image is not suddenly inverted, and changes of combined tomographic images due to movement of the continuous line 61 can be smoothly displayed.

In the ultrasonic measurement apparatus 1 of the present embodiment, in a case where the first point selecting unit 37 selects a first point on the continuous line 61, and an operation signal indicating partial movement is input, the image combining unit 35 divides the continuous line 61 into the first continuous line 611 and the second continuous line 612. The image combining unit 35 displays a combined tomographic image obtained when the first continuous line 611 is rotated centering on the first point 62, and a combined tomographic image obtained when the second continuous line 612 is rotated centering on the first point 62, in real time.

In this case, for example, if a blood vessel branches or bends in the middle, the first point 62 is selected at a position corresponding to a branching point or a bending point, and thus it is possible to acquire a combined tomographic image along a line direction of a blood vessel with high accuracy. Thus, for example, the operator can check whether or not a puncture needle accurately reaches a target position, or a catheter is accurately inserted into a blood vessel after performing puncture work, and thus it is possible to further improve a puncturing success ratio.

The ultrasonic probe 2 of the ultrasonic measurement apparatus 1 of the present embodiment includes the ultrasonic transducers 24 disposed in a two-dimensional array form in the X direction and the Y direction. Among the ultrasonic transducers 24, the ultrasonic transducers 24 disposed in the X direction are connected to each other via the upper electrode 221C3 (common electrode wiring), and are connected to the circuit board 25 from the common terminal 221E1, The ultrasonic transducers 24 disposed in the Y direction are connected to each other via the lower electrode 221C1 (driving electrode line), and are connected to the circuit board 25 from the driving terminal 221D1. When the ultrasonic wave reception process is performed, the voltage source 256 provided on the circuit board 25 outputs the first bias voltage V1 causing reception of an ultrasonic wave to be valid, to the common terminal 221E1 corresponding to the ultrasonic transducers 24 (element portion 23) which are received signal acquisition targets, and outputs the second bias voltage V2 causing reception to be invalid, to the other ultrasonic transducers 24 (element portions 23) which are not received signal acquisition targets. With this configuration, in the ultrasonic transducers 24 other than a region corresponding to acquired internal tomographic images, the reception sensitivity is low, and thus a received signal is reduced to the extent of not influencing measurement thereof . On the other hand, in the ultrasonic transducers 24 corresponding to a region required to form an internal tomographic image, the reception sensitivity is high, and a received signal required to form an internal tomographic image can be appropriately obtained,

Switching sequentially occurs between the common terminals 221E1 which are output destinations of the first bias voltage 1 and the second bias voltage V2, and thus it is possible to switch between the ultrasonic transducers 24 which are received signal acquisition targets. Therefore, a plurality of internal tomographic images along the X direction can be acquired in the Y direction, and thus the living body P can be scanned in a three-dimensional manner. Consequently, in the present embodiment, internal tomographic images can be obtained in a wide range even in a state in which the ultrasonic probe 2 is fixed to the living body P. Therefore, it is possible to reduce time and effort for an operator to adjust a position or an angle of the ultrasonic probe 2. It is also possible to easily acquire a position of a puncture needle in puncture work and thus to considerably reduce a load in the puncture work. Since an operator can concentrate on an operation of the puncture needle 11, it is possible to improve a puncturing success ratio and also to reduce infection disease due to a puncturing failure.

### Second Embodiment

Next, a second embodiment will be described.

In the above-described first embodiment, as illustrated in FIG. 17, in the automatic mode, the continuous line 61 is rotated about each vertex. In contrast, in the second embodiment, movement procedures of the continuous line 61 are different from those in the first embodiment.

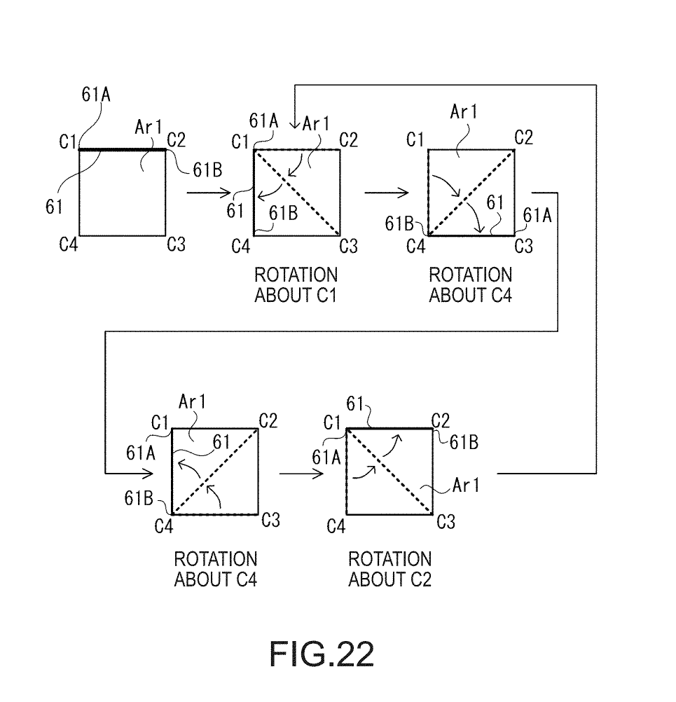

FIG. 22 is a diagram for explaining movement procedures of the continuous line 61 in the second embodiment. In the following description, configurations or steps which have already been described are given the same reference numerals, and description thereof will be omitted or made briefly.

In the present embodiment, the ultrasonic measurement apparatus 1 may be formed of the same configuration as in the first embodiment, and, as illustrated in FIG. 9 or FIG. 19, an ultrasonic measurement process may be performed on the living body P through the substantially same process as in the first embodiment.

In the present embodiment, movement procedures of the continuous line 61 are different from those in the first embodiment in step S8 in FIG. 9.

In other words, in the present embodiment, as illustrated in FIG. 22, the continuous line 61 set in the first vertex C1 and the second vertex C2 is rotated with the first intersection 61A located at the first vertex C1 as the rotation center, and thus the second intersection 61B is moved to the fourth vertex C4 via the third vertex C3.

Next, the first intersection 61A is moved from the first vertex C1 to the third vertex C3 via the second vertex C2 with the second intersection 61B at the fourth vertex C4 as the rotation center.

Next, the rotation direction is inverted, and the first intersection 61A is returned to the first vertex C1, and the second intersection 61B is returned to the second vertex C2. In the ultrasonic measurement apparatus 1 of the present embodiment, the image combining unit 35 performs scanning by rotating the continuous line 61 with two vertices (for example, the first vertex C1 and the fourth vertex C4) which do not have a diagonal relationship therebetween in the rectangular region Ar1 as the rotation center.

Positive or negative of an inclination of the continuous line 61 is the same in rotation about vertices (for example, the first vertex C1 and the third vertex C3) having a diagonal relationship therebetween, and thus detectable line directions of a blood vessel are substantially the same as each other. In this case, an operator can easily determine a line direction of a blood vessel by checking either one of a combined tomographic image (a combined tomographic image obtained when the continuous line 61 is rotated about the first vertex C1 or a combined tomographic image obtained when the continuous line 61 is rotated about the third vertex C3). In other words, in a case where internal tomographic images are displayed by rotating the continuous line with all of the four vertices as the rotation center, a plurality of combined tomographic images which pass through a predetermined single point (s, t) and have the substantially same shape are displayed, and thus a measurement time increases. In contrast, in the present embodiment, combined tomographic images are generated by rotating the continuous line about two vertices having no diagonal relationship therebetween. Also in this c two combined tomographic images passing through a predetermined single point (s, t) are displayed, but positive or negative of an inclination of the continuous line 61 differs in both of the images. Therefore, it is possible to omit waste in measurement time and thus to perform rapid measurement, and also to prevent a decrease in the detection accuracy when a line direction of a blood vessel is detected.

### Third Embodiment

Next, a third embodiment will be described.

In the above-described second embodiment, the continuous line 61 is rotated about the first vertex C1, and then the continuous line 61 is rotated about the fourth vertex C4. In contrast, the third embodiment is different from the second embodiment in that the continuous line 61 is rotated about the first vertex C1, the rotation direction is inverted once, and then the continuous line is rotated.

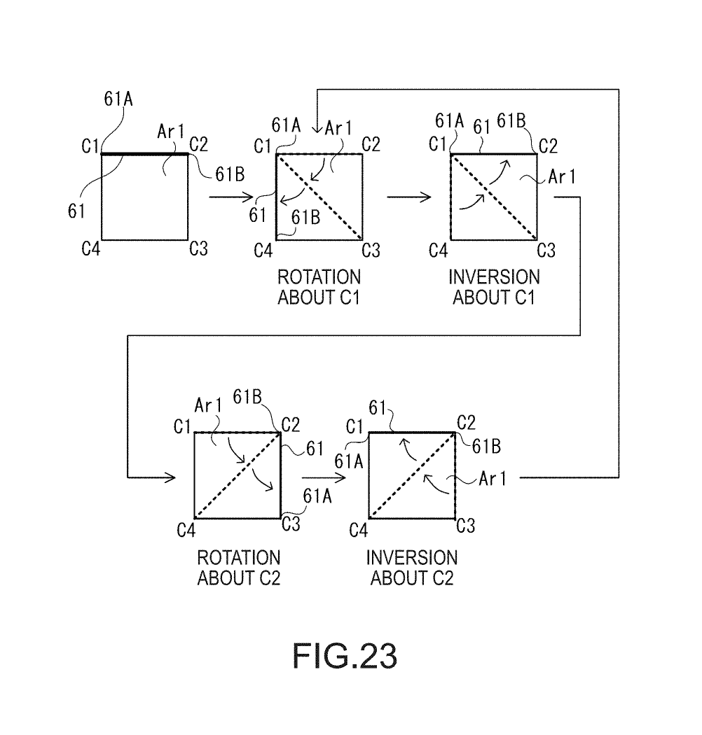

FIG. 23 is a diagram for explaining movement procedures of the continuous line 61 in the third embodiment.

In the present embodiment, the ultrasonic measurement apparatus 1 may be formed of the same configuration as in the first embodiment, and, as illustrated in FIG. 9 or FIG. 19, an ultrasonic measurement process may be performed on the living body P through the substantially same process as in the first embodiment.

In other words, in the present embodiment, as illustrated in FIG. 23, the image combining unit 35 rotates the continuous line 61 set in the first vertex C1 and the second vertex C2 with the first intersection 61A located at the first vertex C1 as the rotation center, and thus the second intersection 61B is moved to the fourth vertex C4 via the third vertex C3. Next, in the present embodiment, the continuous line 61 is inverted with the first intersection 61A located at the first vertex C1 as the rotation center, and the second intersection 61B is returned from the fourth vertex C4 to the second vertex C2 via the third vertex C3.

Thereafter, the image combining unit 35 rotates the continuous line 61 with the second intersection 61B located at the second vertex C2 as the rotation center, and thus moves the first intersection 61A from the first vertex C1 to the third vertex C3 via the fourth vertex C4. The continuous line 61 is inverted with the second intersection 61B located at the second vertex C2 as the rotation center, and the first intersection 61A is returned from the third vertex C3 to the first vertex C1 via the fourth vertex C4.

In the ultrasonic measurement apparatus 1 of the invention, the image combining unit 35 rotates the continuous line 61 from the initial position of the continuous line 61 centering on the first vertex C1, then inverts the continuous line 61 up to the original initial position, and then rotates the continuous line 61 centering on the second vertex C2. in this case, when a combined tomographic image is displayed in an animation manner, a positional relationship between the first intersection 61A and the second intersection 61B viewed from an operator matches a horizontal direction thereof in the combined tomographic image 51 displayed on the display section 4, and thus the operator can easily understand an internal structure of the living body P.

For example, with respect to the rectangular region An in which the first vertex C1 and the fourth vertex C4 are located on the left, and the second vertex C2 and the third vertex C3 are located on the right, when viewed from the operator, the continuous line 61 passing through the first vertex C1 and the second vertex C2 is rotated with the first vertex C1 as the rotation center until the second intersection 61B is located at the fourth vertex C4 via the third vertex C3 in this case, when viewed from the operator, a position of the left first vertex C1 is also displayed to be located on the left in the combined tomographic image 51 on the display section 4, and thus there is no feeling of incompatibility. However, as in the second embodiment, if the first intersection 61A is then moved to the third vertex C3 via the second vertex C2 with the second intersection 61B at the fourth vertex C4 as the rotation center, a position corresponding to the fourth vertex C4 is located, on the right in the combined tomographic image 51 displayed on the display section 4 regardless of the fourth vertex C4 being located on the left when viewed from the operator. Therefore, the left and right sides are inverted to actual ones. In this case, the operator has a feeling of incompatibility for an image, and thus it is hard to understand an internal structure.

In contrast, in the present embodiment, for example, the continuous line 61 passing through the first vertex C1 and the second vertex C2 is rotated with the first vertex C1 as the rotation center until the second intersection 61B is moved to the fourth vertex C4, the rotation direction is inverted, and the second intersection 61B is returned to the second vertex C2. Thereafter, the continuous line 61 is rotated with the second intersection 61B at the second vertex C2 as the rotation center until the first intersection 61A is moved to the third vertex C3. In this case, a position of the combined tomographic image 51 displayed on the display section 4 and an actual position of the continuous line 61 are not horizontally inverted, and thus it is possible to easily understand a position of the continuous line 61 with respect to the combined tomographic image 51. Consequently, the operator can appropriately understand an internal structure of the living body P. Therefore, it is possible to efficiently perform puncture work and also to improve a puncturing success ratio.

### Modification Examples

Each of the above-described embodiments is only an example, and configurations obtained through modifications, alterations, and combinations of the respective embodiments within the scope of being able to achieve the object thereof are included in the invention.

In the first embodiment, a description has been made of an example in which the image combining unit 35 rotates the continuous line 61 about each of the first vertex C1 to the fourth vertex C4, but any other method may be used.

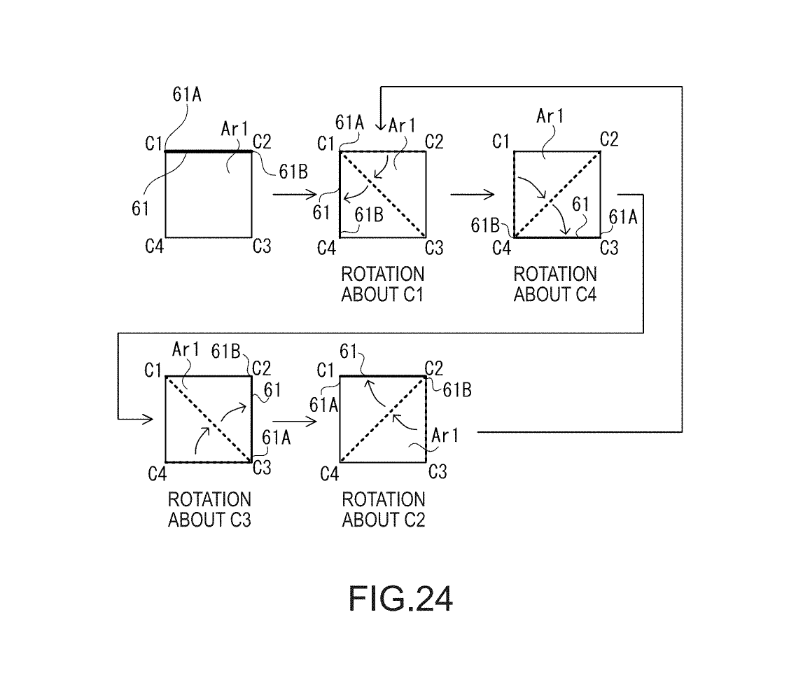

FIG. 24 is a diagram illustrating other examples of movement procedures of the continuous line 61.

As illustrated in FIG. 24, the image combining unit 35 rotates the continuous line 61 set in the first vertex C1 and the second vertex C2 with the first intersection 61A located at the first vertex C1 as the rotation center, and thus moves the second intersection 61B to the fourth vertex C4 via the third vertex C3.

Next, the image combining unit 35 rotates the continuous line 61 with the second intersection 61B located at the fourth vertex C4 as the rotation center until the first intersection 61A is moved from the first vertex C1 to the third vertex C3 via the second vertex C2.

Next, the image combining unit 35 rotates the continuous line 61 with the first intersection 61A located at the third vertex C3 as the rotation center until the second intersection 61B is moved from the fourth vertex C4 to the second vertex C2 via the first vertex C1.

The image combining unit 35 rotates the continuous line 61 with the second intersection 61B located at the second vertex C2 as the rotation center until the first intersection 61A is moved from the third vertex C3 to the first vertex C1 via the fourth vertex C4.

In this case, image inversion occurs, but a combined tomographic image obtained when the continuous line 61 is rotated by 90 degrees about each vertex can be displayed, and thus an operator can more easily detect a line direction of a blood vessel.

The first embodiment or the example illustrated in FIG. 24 relates to an example in which the continuous line is rotated with each vertex as the rotation center, and the second embodiment or the third embodiment relates to an example in which the continuous line is rotated with two vertices having no diagonal relationship therebetween as the rotation center, but the continuous line may be rotated with three vertices as the rotation center 25 is a diagram illustrating still other examples of movement procedures of the continuous line 61.

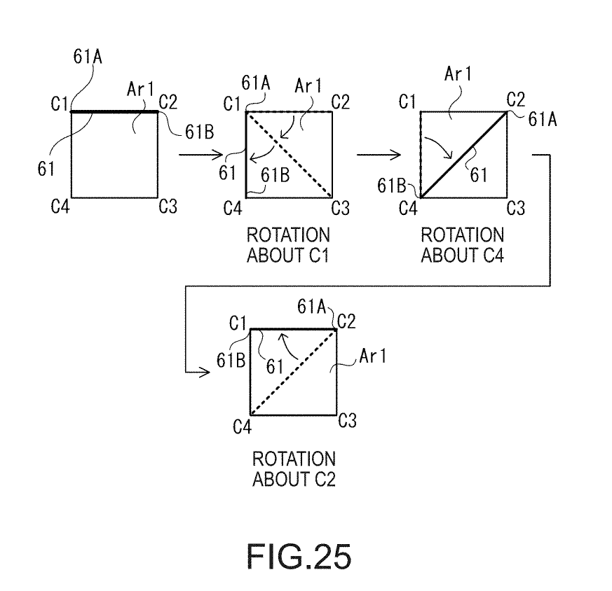

In an example illustrated in FIG. 25, the image combining unit 35 rotates the continuous line 61 set in the first vertex C1 and the second vertex C2 with the first intersection 61A located at the first vertex C1 as the rotation center, and thus moves the second intersection 61B to the fourth vertex C4 via the third vertex C3.

Next, the image combining unit 35 rotates the continuous line 61 with the second intersection 61B located at the fourth vertex C4 as the rotation center until the first intersection 61A is moved from the first vertex C1 to the second vertex C2.

Next, the image combining unit 5 rotates the continuous line 61 with the first intersection 61A located at the second vertex C2 as the rotation center until the second intersection 61B is moved from the fourth vertex C4 to the first vertex C1. In this case, positions of the first intersection 61A and the second intersection 61B are inverted with respect to the initial continuous line 61, and the combined tomographic image 51 is also displayed on the display section 4 in a form of being horizontally inverted, but the continuous line 61 can be returned to the original position by performing the same operation again.

There may be a configuration in which an operator selects the movement procedures in the first to third embodiments, and the movement procedures as illustrated in FIG. 24 or 25 as appropriate, and there may be a configuration in which the operator can set and input a movement destination of the first intersection 61A or the second intersection 61B.

In the above-described respective embodiments, a description has been made of an example in which the continuous line 61 is straight line, but the continuous line 61 is not limited thereto. The continuous line may be, for example, a circular arc line or a dashed line. Particularly, in the manual mode, an operator manually inputs an expression representing a continuous line and can thus set a continuous line having any shape.

In the second ultrasonic measurement process of the first embodiment, in a case where a determination result is negative (No) in step S22, only the second continuous line 612 is rotated, and the first continuous line 611 is rotated in a state in which the second continuous line 612 is fixed, but any other method may be used. For example, the first continuous line 611 may be rotated, and then the second continuous line 612 may be rotated.

There may be a configuration in which an operator can select a line which will be first rotated of the first continuous line 611 and the second continuous line 612.

Of the first continuous line 611 and the second continuous line 612, one line having a shorter length dimension is first rotated, and then the other line may be rotated.

In the first embodiment, a description has been made of an example in which a single first point 62 is selected, but any other method may be used. By repeatedly performing the second ultrasonic measurement process, the number of first points selected by the first point selecting unit 37 can be increased, In this case, it is possible to appropriately generate a combined tomographic image along a line direction of a blood vessel with respect to a blood vessel having a complex branching structure or a blood vessel having a lot of bending points. In the respective embodiments, a description has been made in which the X direction (first direction) and the Y direction (second direction) are orthogonal to each other, but the two directions may not necessarily be orthogonal to each other as long as the directions intersect each other. For example, an angle of 60 degrees may be formed between the X direction and the Y direction.

In the above-described embodiments, the display control unit 38 also functions as a section position display unit, and displays the combined tomographic image 51 and the section position image 53 indicating a position of the continuous line 61 in an arranged manner on the display section 4, but is not limited thereto.

For example, the display control unit 38 may display the section position image 53 by changing display on the display section 4 in response to an input operation from an operator in a state in which the combined tomographic image 51 is displayed.

For example, a display may be performed on an upper surface of the ultrasonic probe 2, and the section position image 53 may be displayed on the display. In other words, a position of the continuous line 61 corresponding to the combined tomographic image 51 displayed on the display section 4 is directly displayed on the ultrasonic probe 2. In this case, an operator can more easily understand a position of the continuous line 61 corresponding to the combined tomographic image 51 displayed on the display section 4, and thus it is possible to more efficiently perform puncture work and also to improve a puncturing success ratio.

In the first embodiment, as an example, the image selecting unit 36 selects an image on the basis of an input operation from an operator, but is not limited thereto. For example, the image selecting unit 36 may perform image analysis on each generated combined tomographic image so as to specify a blood vessel, and may perform a process of selecting a combined tomographic image in which a dimension of the blood vessel in a long axis direction is the maximum.

The image selecting unit 36 may not be provided. In the above-described embodiments, since the continuous line 61 is automatically moved, and thus combined tomographic images are displayed in a switching manner in real time, it may be effective for the image selecting unit 36 to select an image. However, for example, in a case where respective combined tomographic images obtained when the continuous line 61 is moved are displayed in a list form on the display section 4, the combined tomographic images at the respective time points may be displayed on the display section 4 in a list form without selecting an image. In this case, even if the image selecting unit 36 is not provided, an operator can find a desired image from the combined tomographic images 51 displayed on the display section 4 in a list form.

In the first embodiment, as an example, the element board 221 has a configuration in which the opening 221A1 corresponding to each ultrasonic transducer 24 is provided in the base 221A, but is not limited thereto. The opening 221A1 defines a vibration region of the vibration film 221B in the ultrasonic wave transmission process or the ultrasonic wave reception process, and is not limited to the opening 221A1 surrounded by the partition walls 221A2. For example, a rectangular opening 221A1 may be provided in the base 221A in the Y direction, and the piezoelectric element 221C may be disposed on the vibration film 221B closing the opening 221A1 in the Y direction. A bonding portion which bonds the vibration film 221B and the sealing plate 222 together may be provided between the respective piezoelectric elements 221C. In this configuration, a vibration region of the vibration film 221B in a single ultrasonic transducer 24 can be defined by the partition wall 221A2 of the base 221A forming the opening 221A1 and the bonding portion. A size of the opening 221A1 can be relatively increased, and thus the manufacturing efficiency of the ultrasonic sensor 22 can be improved.

In the above-described first embodiment, in the ultrasonic sensor 22, a description has been made of an example in which an ultrasonic wave is transmitted from the opening 221A1 side of the base 221A, and an ultrasonic wave which is input to the vibration film 221B from the opening 221A1 is received, but any other configuration may be employed. For example, there may be a configuration in which, in the ultrasonic sensor, the sealing plate 222 is bonded to the opening 221A1 side of the base 221A, an ultrasonic wave is transmitted from the vibration film 221B side, and an ultrasonic wave which is input from the vibration film 221E is received.

In the first embodiment, a description has been made of an example in which the ultrasonic transducer 24 is formed of the vibration film 221B closing the opening 221A1 of the base 221A and the piezoelectric element 221C, but any other configuration may be employed.

For example, there may be a configuration in which a vibration film may be disposed on a board via an air gap, and opposing electrodes with the air gap interposed therebetween may be disposed on the board and the vibration film. In this case, there may a configuration in which an electrostatic attraction force is caused by the electrodes by outputting a periodic drive signal between the electrodes, and thus the vibration film vibrates.

In the above-described respective embodiments, a scanning process is performed in the X direction in order to acquire an internal tomographic image along the X direction, and received signals corresponding to a plurality of internal tomographic images are acquired by deviating a position where the scanning process is performed in the Y direction, In contrast, for example, there may be a configuration in which a scanning process is performed in the Y direction, received signals for respective measurement regions are acquired by deviating a position where the scanning process is performed in the X direction, and an internal tomographic image in the X direction is formed by combining the received signals for the respective measurement regions in the X direction with each other.

The respective embodiments and modification examples may be combined with each other as appropriate within the scope of being capable of achieving the object of the invention, and may be altered to other structures as appropriate,

The entire disclosure of Japanese Patent Application No. 2016-046653 filed Mar. 10, 2016 is expressly incorporated by reference herein.

What is claimed is:

1. An ultrasonic image processing device comprising:
an image acquisition unit that acquires a plurality of internal tomographic images of a target object in a plane including a first direction, along a second direction intersecting the first direction;
an image dividing unit that divides each of the internal tomographic images into a plurality of separate images with a normal line to the first direction, and acquires the separate images; and
an image combining unit that extracts separate images corresponding to coordinates on a continuous line which is continued in a plane including the first direction and the second direction, from the plurality of separate images, and arranges and combines the separate images in order of coordinates along the continuous line so as to generate a combined tomographic image.

2. The ultrasonic image processing device according to claim 1,
wherein the continuous line is a straight line.

3. The ultrasonic image processing device according to claim 1, further comprising:
a display control unit that displays the combined tomographic image combined by the image combining unit on a display section,
wherein the image combining unit generates a plurality of the combined tomographic images obtained when the continuous line is moved in a plane including the first direction and the second direction, and
wherein the display control unit displays the plurality of combined tomographic images on the display section.

4. The ultrasonic image processing device according to claim 3, further comprising:
an image selecting unit that selects a predetermined combined tomographic image from among the plurality of combined tomographic images,
wherein the display control unit displays the combined tomographic image selected by the image selecting unit.

5. The ultrasonic image processing device according to claim 4, further comprising:
a section position display unit that displays a position of the continuous line corresponding to the combined tomographic image selected by the image selecting unit.

6. The ultrasonic image processing device according to claim 3,
wherein the image combining unit generates the plurality of combined tomographic images obtained when the continuous line passing through a first point in a plane including the first direction and the second direction is rotated centering on the first point.

7. The ultrasonic image processing device according to claim 6,
wherein the first point is a vertex of a predetermined rectangular region in the plane including the first direction and the second direction.

8. The ultrasonic image processing device according to claim 7,
wherein the image combining unit generates the plurality of combined tomographic images obtained when the continuous line is rotated with each vertex of the rectangular region as the first point.

9. The ultrasonic image processing device according to claim 7,
wherein the image combining unit generates the plurality of combined tomographic images obtained when the continuous line is rotated with, as the first point, two vertices having no diagonal relationship therebetween among vertices of a predetermined rectangular region in the plane including the first direction and the second direction.

10. The ultrasonic image processing device according to claim 7,
wherein, in a case where intersections between the continuous line and an outer peripheral edge of the rectangular region are set as a first intersection and a second intersection, the image combining unit rotates the continuous line by alternately replacing the first point with the first intersection and the second intersection.

11. The ultrasonic image processing device according to claim 10,
wherein the image combining unit rotates the continuous line and then inverts a rotation direction.

12. The ultrasonic image processing device according to claim 6, further comprising:
a first point selecting unit that selects the first point on the continuous line,
wherein, in a case where the first point is not an end of the continuous line, the image combining unit generates the plurality of combined tomographic images obtained when one of separate lines obtained by dividing the continuous line with respect to the first point is rotated centering on the first point.

13. An ultrasonic measurement apparatus comprising:
an ultrasonic probe that acquires a plurality of internal tomographic images of a target object in a plane including a first direction, along a second direction intersecting the first direction through transmission and reception of an ultrasonic wave;
an image acquisition unit that acquires the internal tomographic images from the ultrasonic probe;
an image dividing unit that divides each of the internal tomographic images into a plurality of separate images with a normal line to the first direction, and acquires the separate images; and
an image combining unit that extracts separate images corresponding to coordinates on a continuous line which is continued in a plane including the first direction and the second direction, from the plurality of separate images, and arranges and combines the separate images in order of coordinates along the continuous line so as to generate a combined tomographic image.

14. The ultrasonic measurement apparatus according to claim 13,
wherein the ultrasonic probe includes
a plurality of ultrasonic transducers that are disposed in an array form along the first direction and the second direction;
a common electrode wiring that connects the ultrasonic transducers along the first direction to each other;
a driving electrode wiring that connects the ultrasonic transducers along the second direction to each other; and
a bias voltage output portion that outputs a bias voltage to the common electrode wiring, and
wherein the bias voltage output portion includes a voltage switching unit that switches between a first bias voltage causing reception of the ultrasonic wave to be valid and a second bias voltage causing reception of the ultrasonic wave to be invalid.

15. An ultrasonic image processing method comprising:
acquiring a plurality of internal tomographic images of a target object in a plane including a first direction, along a second direction intersecting the first direction;
dividing each of the internal tomographic images into a plurality of separate images with a normal line to the first direction, and acquiring the separate images; and
extracting separate images corresponding to coordinates on a continuous line which is continued in a plane including the first direction and the second direction, from the plurality of separate images, and arranging and combining the separate images in order of coordinates along the continuous line so as to generate a combined tomographic image.
